(19) United States
(12) Patent Application Publication
(10) Pub. No.: US 2025/0263686 A1
(43) Pub. Date: Aug. 21, 2025

(54) METHODS OF INHIBITING NUCLEASE ACTIVITY, METHODS OF ISOLATING NUCLEI FROM CELLS, AND METHODS FOR EXTENDING DNA

(71) Applicant: NATIONAL CHENG KUNG UNIVERSITY, Tainan City (TW)

(72) Inventors: Po-Min CHIANG, Tainan City (TW); Ya-Wen HSUEH, Tainan City (TW)

(21) Appl. No.: 18/951,692

(22) Filed: Nov. 19, 2024

Related U.S. Application Data
(60) Provisional application No. 63/555,937, filed on Feb. 21, 2024.

(51) Int. Cl. C12N 15/10

(52) U.S. Cl. CPC C12N 15/1006

(57) ABSTRACT

Disclosed herein is a method of inhibiting the activity of a nuclease in a biological sample. According to some embodiments of the present disclosure, the method comprises mixing the biological sample with a metal ion-chelator complex. Also disclosed herein is a method of isolating nuclei from cells in a biological sample by using the metal ion-chelator complex, and a method of adding a deoxyribonucleotide to the 3′ end of a deoxyribonucleic acid (DNA).

### CROSS-REFERENCE TO RELATED APPLICATIONS

This application relates to and claims the benefits of U.S. Provisional Application No. 63/555,937 filed Feb. 21, 2024; the content of the application is incorporated herein by reference in the entirety.

### SEQUENCE LISTING XML

The present application is being filed along with a Sequence Listing XML in electronic format. The Sequence Listing XML is provided as a XML file entitled P4406_SEQ_AF, created Aug. 21, 2024, which is 24 Kb in size. The information in the electronic format of the Sequence Listing XML is incorporated herein by reference in its entirety.

### BACKGROUND OF THE INVENTION

### 1. Field of the Invention

The present disclosure in general relates to the field of molecular biology. More particularly, the present disclosure relates to methods of inhibiting nuclease activity, methods of isolating nuclei from biological samples, and methods for extending DNA.

### 2. Description of Related Art

Nucleic acid amplification is crucial for achieving high detection sensitivity in clinical and biological samples. Currently, amplification involves two major strategies: in vitro transcription and polymerase chain reaction (PCR). In vitro transcription, in addition to requiring vulnerable cRNA intermediates and facing inefficiencies in dsDNA synthesis, also involves tedious multi-step purifications, which can lead to inevitable sample loss. In contrast, PCR amplification is a more common and straightforward technique, as it typically involves fewer steps and avoids RNA production. However, PCR requires adding a handler to the 3′ end of DNA. This can be achieved through methods such as tagmentation following dsDNA synthesis, polymerization using handler-bearing randomers, template-switching, or the use of homopolymers. Tagmentation following double-stranded deoxyribonucleic acid (dsDNA) synthesis suffers from inefficiencies in dsDNA synthesis and sequence bias during the tagmentation process. Polymerization using handler-bearing randomers also involves biases due to the preference for randomer binding, secondary structures of the target DNA, and the need to remove these primers to avoid interference with subsequent PCR amplification. The single-step addition of the PCR handler through template-switching or homopolymerization, followed by purification-free amplification, maximizes detectable DNA fragments. Nonetheless, template-switching is not 100% efficient due to potential non-dC nucleotide additions to DNA tails. Homopolymerization using terminal deoxynucleotidyl transferase (TdT) is inefficient with all current reaction systems. More specifically, it is currently impossible to completely extend recessed DNA ends, such as those found on complementary mRNA or embedded in secondary structures. Thus, it is highly desirable to identify and develop a reaction system capable of fully tailing all DNA substrates, especially those with recessed ends, to enable purification-free DNA amplification.

Nucleases are ubiquitous in environments and biological samples, degrading and hindering the detection of nucleic acids for clinical and research purposes. Ribonuclease (RNase) is one of the most challenging nucleases to handle due to its ubiquitous presence, metal-ion independence, and resistance to heat inactivation. Current strategies for blocking RNase are associated with significant limitations, particularly when applied to delicate samples such as nuclei in tissues: the use of low-pH strategies denatures proteins, alters nuclear architecture, and degrades nucleic acids. Vanadyl ribonucleoside depends on divalent cations and potentially interferes downstream enzymatic activities. Similar to recombinant RNase inhibitors, this nucleoside analogue is difficult to acquire in large amounts and is ineffective against samples containing various types of nucleases, particularly DNases. Reducing agents require prolonged or high-temperature incubation, which is incompatible with one-pot lysis, where RNAs are immediately exposed to nucleases. The use of highly negatively charged polymers, such as polyvinyl sulfonic acid, to bind and inactivate RNases also competitively binds positively charged nuclear proteins and distorts the structure of unfixed nuclei. Furthermore, these large polymers are difficult to remove from the extracted nuclei, which complicates downstream enzymatic reactions. Finally, diethyl pyrocarbonate (DEPC) carboxymethylates RNA purines and readily reacts with amino acid side chains, particularly lysine, competing with crosslinkers such as formaldehyde and NHS esters for efficient crosslinking. Although Cu²⁺ has been shown to inhibit RNase activity, it is not suitable for most applications in biological systems: (1) the ion forms insoluble precipitates (Cu(OH)₂) at neutral pH; (2) it crosslinks proteins by interacting with multiple histidine residues, leading to protein coagulation. These limitations render Cu²⁺ alone unusable as a RNase or nuclease inhibitor because the concentrations required to effectively block hydrolytic activity are much higher than the soluble Cu²⁺ present in almost all reaction systems. Collectively, a non-coagulating, soluble agent that can protect nucleic acids, including RNA and DNA, in biological samples without significantly altering the native environment (such as pH, ionic conditions, and temperature) or causing unnecessary covalent modifications, and that is also easy to remove, remains highly desirable but is not yet available.

One increasingly important objective is to profile nucleic acid levels in individual cells and nuclei. Cellular RNA and DNA are exposed to nucleases present in cellular vesicles and extracellular fluids, such as plasma, in multicellular organisms immediately upon lysis. While it's feasible to dissociate individual cells from tissues for intact-cell profiling, tissue-dependent procedures introduce unavoidable artifacts, hindering cross-tissue comparisons of the same cell types. In addition, the dissociation potentially leads to biased release of different cell types and the ex vivo incubation is also associated with cell state changes. Given the proper sensitivity and the ability to classify cell type using nuclei as input, directly isolating nuclei from pulverized tissues can circumvent the issues associated with cell dissociation mentioned earlier. This approach also substantially expands the acceptable sample range. However, the abundant nucleases in certain tissues, such as the blood-rich spleen or exocrine pancreas, will quickly degrade nucleic acids in the nuclei within the lysate. This difficulty is evident from the absence of one-pot lysis datasets for the spleen and the need for a special low-pH buffer for pancreatic acinar cells.

In view of the foregoing, there is a continued interest in developing novel methods for protecting nucleic acids in biological samples, particularly RNA in nuclei, and for efficiently amplifying the inherent or converted DNA, especially those with inaccessible recessed ends, to enable sensitive detection.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the present disclosure is directed to a method of inhibiting the activity of a nuclease, including a deoxyribonuclease (DNase) and ribonuclease (RNase), in a biological sample. According to some embodiments of the present disclosure, the method comprises mixing the biological sample with a metal ion-chelator complex so as to inhibit the activity of the nuclease in the biological sample.

According to certain embodiments of the present disclosure, the metal ion-chelator complex is copper citrate (Cu-Citrate), copper nitrilotriacetic acid (Cu-NTA), or copper iminodiacetic acid (Cu-IDA).

Preferably, the molar ratio of the metal ion and chelator in the complex is greater or equals to 1. More preferably, the molar ratio of the metal ion and chelator in the complex equals to 1.

The second aspect of the present disclosure pertains to a method of isolating nuclei from cells in a biological sample by using the metal ion-chelator complex. According to some embodiments of the present disclosure, the method comprises,
(a) mechanically disrupting the cells in the biological sample;
(b) mixing the product of step (a) with a lysis buffer in the presence of the metal ion-chelator complex (e.g., Cu-Citrate, Cu-NTA, or Cu-IDA) so as to release the nuclei from the cells; and
(c) separating the nuclei from the product of step (b) thereby producing the isolated nuclei.

According to certain exemplary embodiments of the present disclosure, in the step (c) of the present method, the nuclei are separated by steps of,
(c-1) subjecting the product of step (b) to density-gradient centrifugation in the presence of the metal ion-chelator complex; and
(c-2) collecting the fraction containing the nuclei from the product of step (c-1).

According to various embodiments, the lysis buffer of step (b) comprises 0.1-3% (v/v) nonionic detergent. In one specific embodiment, the lysis buffer of step (b) comprises 0.1% (v/v) polyoxyethylene sorbitan monolaurate (polysorbate 20) and 0.1% (v/v) nonyl phenoxypolyethoxylethanol (NP-40).

According to certain preferred embodiments, the metal ion-chelator complex is the Cu-Citrate, which is present in the lysis buffer at a concentration of 2-100 mM. In one specific example, the Cu-Citrate is present in the lysis buffer at a concentration of 10 mM.

According to some embodiments, the method further comprises:
(d) fixing the nuclei of step (c) with a solution comprising methanol and magnesium ions (Mg²⁺). In some preferred embodiments, the Mg²⁺ ions are present in the solution at a concentration of 5 mM.

The third aspect of the present disclosure pertains to a method of adding a deoxyribonucleotide to the 3′ end of a deoxyribonucleic acid (DNA). The method comprises,
(a) mixing the DNA with a reaction buffer comprising a terminal deoxynucleotidyl transferase (TdT), the deoxyribonucleotide, and a transition metal ion, wherein the reaction buffer is free of ions other than the transition metal ion (such as free of potassium ions (K⁺), magnesium ions (Mg²⁺), sodium ions (Na⁺), etc.); and
(b) incubating the mixture of step (a) at 37° C. for 30-120 minutes thereby producing the DNA having the deoxyribonucleotide added to its 3′-end.

According to certain embodiments of the present disclosure, the transition metal ion is cobaltous ion (Co²⁺) or manganous ion (Mn²⁺).

According to some embodiments of the present disclosure, the deoxyribonucleotide is deoxyadenosine triphosphate (dATP), deoxycytidine triphosphate (dCTP), deoxythymidine triphosphate (dTTP), or deoxyuridine triphosphate (dUTP). According to certain exemplary embodiments, the deoxyribonucleotide is dATP, and the concentration of the dATP in the reaction buffer ranges from 0.5 mM to 10 mM. In one specific embodiment, the concentration of the dATP in the reaction buffer is 2 mM.

Depending on desired purpose, the DNA may be a double-strained DNA or a single-strained DNA (e.g., a cDNA). According to some embodiments, the DNA is a double-stranded DNA with a 3′ recessed end.

The fourth aspect of the present disclosure provides a method of analyzing the expression of a plurality of mRNAs in cells of a biological sample. The method comprises,
(a) mechanically disrupting the cells in the biological sample;
(b) mixing the product of step (a) with a lysis buffer in the presence of the present metal ion-chelator complex (e.g., Cu-Citrate, Cu-NTA, or Cu-IDA) so as to release the nuclei from the cells;
(c) separating the nuclei from the product of step (b);
(d) fixing the nuclei of step (c) with a solution comprising methanol and magnesium ions (Mg²⁺);
(e) mixing the product of step (d) with a reverse transcription (RT) buffer comprising a reverse transcriptase, deoxyribonucleotide triphosphates (dNTPs), and a plurality of barcoded RT primers;
(f) subjecting the mixture of step (e) to reverse transcription (RT) reaction thereby producing a first plurality of cDNAs, each of which comprises a first unique barcode sequence;
(g) mixing the first plurality of cDNAs of step (f) with a reaction buffer comprising a terminal deoxynucleotidyl transferase (TdT), deoxyadenosine triphosphate (dATP), and cobaltous ions (Co²⁺) or manganous ions (Mn²⁺), wherein the reaction buffer is free of potassium ions (K⁺), magnesium ions (Mg²⁺) and sodium ions (Na⁺);
(h) incubating the mixture of step (g) at 37° C. for 30-120 minutes thereby producing a plurality of poly-adenylated cDNAs;
(i) producing a plurality of double-stranded cDNAs via subjecting the product of step (h) to a polymerase chain reaction (PCR);
(j) sequencing the product of step (i) so as to determine the number of each unique barcode sequence; and
(k) determining the expression of each mRNA based on the result of step (j).

According to some embodiments, in step (i) of the present method, the plurality of double-stranded DNAs are produced by,
(i-1) mixing the product of step (h) with a PCR buffer comprising an oligo(dT) primer, a DNA polymerase and dNTPs; and
(i-2) subjecting the product of step (i-1) to the PCR.

Optionally, the method further comprises amplifying the product of step (i) prior to step (j).

Optionally, the method further comprises the following steps prior to step (g),
(f-1) mixing the first plurality of cDNAs of step (f) with a ligation buffer comprising a DNA ligase and a plurality of adapters, wherein each of the plurality of adapters comprises a second unique barcode sequence; and
(f-2) incubating the mixture of step (f-1) at 37° C. for 30-120 minutes thereby producing a second plurality of cDNA, each of which comprises the second unique barcode sequence linked to the 3′ end of the first unique barcode sequence.

Still optionally, the method further comprises repeating steps (f-1) and (f-2) at least once prior to step (g).

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings.

FIG. 1 is a photograph of silver staining depicting TdT activities in specified reaction conditions according to Example 1 of the present disclosure, in which an ssDNA oligonucleotide (SEQ ID NO: 10) was incubated with specified agents in the presence with dATP (2 mM) and commercial TdT (0.25 μL). THERMOPOL®: THERMOPOL® reaction buffer; K⁺: potassium acetate; Mg²⁺: magnesium acetate; Co²⁺: cobalt chloride. Tris-acetate pH7.9 (20 mM) and TRITON® X-100 (0.1%) were included in the reactions of lanes 3-8. The arrow denotes the untailed oligo substrates.

FIG. 2 is a photograph of silver staining depicting the effects of divalent cations and substrate amounts on tailing a recessed-end DNA substrate according to Example 2 of the present disclosure. The substrate was incubated in reaction mixtures in the presence of Mg²⁺ or Co²⁺ as tailing cofactors. The 5,000 cell-lysate (Lysate+) and lysis buffer-only (Lysate−) from fetal liver mocked the presence and absence of background DNA ends, respectively. The arrows denote the untailed oligo substrates.

FIG. 3 is a photograph of silver staining depicting the effects of deoxyribonucleotide (dNTP), buffer, reducing agent (dithiothreitol (DTT)), and ion/ion concentration (M²⁺/concentration) on tailing the recessed-end DNA substrate according to Example 3 of the present disclosure. The arrows denote the untailed oligo substrates.

FIG. 4 is a photograph of silver staining depicting the effects of TdT, substrates, and cofactors on the tailing efficiency according to Example 4 of the present disclosure. The arrows denote the untailed oligo substrates.

FIG. 5 is the result of fluorescent assay depicting the effect of Zn²⁺ (Zn) and Cu²⁺ (Cu) on inhibiting pancreatic nucleases according to Example 5 of the present disclosure. Lysate denotes 4 μL of crude lysate from the murine pancreas (+) or the lysis buffer-only control (−). Lane 2 in squares and 1 in circles served as nuclease-positive and -negative controls, respectively. **** denotes p<0.0001 based on one-way ANOVA with Dunnett post hoc tests using lane 2 as the reference.

FIG. 6 depicts optical density measurements at 600 nm of Cu²⁺ (triangles), Cu²⁺ plus bovine serum albumin (squares), and Cu²⁺ plus bovine serum albumin and the chelator citrate according to Example 6 of the present disclosure. The buffers used for the respective pH levels are as follows: pH 4.5: HEPES-SO₄, pH 5.5: MES (2-(N-morpholino)ethanesulfonic acid), and pH 7.2: HEPES (4-(2-Hydroxyethyl)piperazine-1-ethanesulfonic acid, N-(2-Hydroxyethyl)piperazine-N′-(2-ethanesulfonic acid)).

FIG. 7 is the result of a fluorescent assay depicting the effects of Cu²⁺ alone (squares) and Cu²⁺ in complex with the chelator citrate (circles) on pancreatic nucleases activity, as described in Example 7 of the present disclosure. The symbol # indicates the presence of precipitation.

FIG. 8 is the result of fluorescent assay depicting the effects of pH, Cu²⁺ in complex with different chelators, and chelators alone on inhibiting pancreatic nucleases according to Example 8 of the present disclosure. CuN (Cu²⁺-NTA), CuC (Cu²⁺-citric acid), and C (sodium citrate) were respectively prepared from 250 mM concentrated stock solutions, with pH adjusted to 4.5 (Panel (A)) or 7.2 (Panel (B)). Lysate denotes 1.5 μL of crude lysate from the murine pancreas (+) or the lysis buffer-only control (−). **** denotes p<0.0001 based on one-way ANOVA with Dunnett post hoc tests using lanes 2 in respective panels as the reference.

FIG. 9 is the result of fluorescent assay depicting the blocking effects of Cu²⁺-citric acid complex on different kind of nucleases, including RNase A (50 ng), micrococcal nuclease (200 gel units), and benzonase (Golden nuclease, 20 units) according to Example 9 of the present disclosure. CuC: Cu²⁺-citric acid pH 7.2. **** denotes p<0.0001 based on one-way ANOVA with Dunnett post hoc tests using corresponding no-nuclease samples as the reference.

FIG. 10 is the result of fluorescent assay depicting the effects of chelators NTA, citrate, and EDTA (ethylenediaminetetraacetic acid) in the presence of Cu²⁺ on inhibiting pancreatic nucleases according to Example 10 of the present disclosure. **** denotes p<0.0001 based on one-way ANOVA with Dunnett post hoc tests using lanes 2 as the reference.

FIG. 11 is the result of fluorescent assay depicting the effect of Cu-Citrate (CuC), along with two other inhibitory treatment (DEPC and low pH) on inhibiting pancreatic nucleases according to Example 11 of the present disclosure. DEPC: diethyl pyrocarbonate. N: neutral buffer. C: the low pH buffer (25 mM citrate at pH 3 and 250 mM sucrose). Lysate: 4 μL of crude lysate from the murine pancreas (+) or the lysis buffer-only control (−). Lane 2 and lane 1 respectively served as nuclease-positive and -negative controls. **** denotes p<0.0001 based on one-way ANOVA with Dunnett post hoc tests using lanes 2 as the reference.

FIG. 12 is the result of a reverse-transcription quantitative polymerase chain reaction (RT-qPCR) depicting the protection of nuclear mRNA in the presence of pancreatic nuclease by the metal-chelator complex according to Example 12 of the present disclosure. HEK293 nuclei were incubated with the complex (presence, +) or without it (absence, −) at pH 4.5 (squares) or 7.2 (circles) and subjected to RT-qPCR to assess the relative enrichment of two housekeeping genes, GAPDH and PSMB4, as compared to the genomic DNA control (dashed lines). *, **, and denote p-values of <0.05, <0.01, and <0.001, respectively, based on Student's t-tests.

FIG. 13 is a boxplot depicting the number of genes detected at 20,000 subsampled reads per cell in HEK293 cells across various technologies, as described in Example 13. The four methods that achieved high cell capacities comparable to USPPAR were marked as ‘high’, while the remaining methods offered lower cell capacity (marked as ‘low’) per experiment.

FIG. 14 depicts the violon plot of gene detection efficiencies of the B cells prepared with the snRNA-seq based on this invention (USPPAR) and the commercial scRNA-seq platform (10×_v3), as described in Example 14. For plotting, 2,500 reads were subsampled from each barcode/cell. The p-value was obtained using the Wilcoxon rank-sum test.

FIG. 15 depicts the gene-detection efficiencies of maize shoot nuclei by snRNA-seq using USPPAR (USPPAR) and 10× Chromium (10×_v3) as described in Example 15. Median numbers of reads (solid), UMIs (dashed), and genes (dotted) per nucleus are plotted at down-sampled sequencing depths of 500, 1,000, 2,500, 5,000, and 10,000. For the USPPAR dataset, all barcodes containing >400 genes were kept. For the reference dataset, only high-quality barcodes containing >500 genes and with cell-type annotations were kept, as in the original reference.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

As used herein, the term “complex” refers to an assemblage or aggregate of two or more molecules in direct and/or indirect contact with one another. In the present disclosure, the term “metal ion-chelator complex” refers to a substance formed by associated of a metal ion and a chelator that are bonded via covalent bonds.

The term “chelator” as used herein refers to a molecule capable of linking or binding with a metal ion by forming multiple bonds to the metal ion. Depending on desired purpose, the chelator suitable to form the present metal ion-chelator complex may be a biological molecule (such as hemoglobin, transferrin, lactoferrin, conalbumin, or ferritin), or an organic molecule (such as EDTA, citrate, NTA, IDA, diethylenetriamine, ethylenediamine, N,N′,N″-tris(2-pyridylmethyl)-1,3,5-cis,cis-triaminocyclohexane (tachpyr), or other molecules identifiable to a skilled person).

As used herein, the term “nonionic detergent” refers to a class of detergents that do not have ionic groups and thus would not ionize in aqueous solutions; for example, nonyl phenoxypolyethoxylethanol (also known as “NP-40”), TRITON® X-100, polyoxyethylene octylphenyl etherpolyoxyethylene (also known as “IGEPAL® CA-720”), octylphenoxy poly(ethyleneoxy)ethanol (also known as “IGEPAL® CA-630”), polyoxyethylene sorbitan monolaurate (also known as “polysorbate 20” or “TWEEN® 20”), polyoxyethylene sorbitan monooleate (also known as “polysorbate 80” or “TWEEN® 80”), polyoxyethylene sorbitan trioleate (also known as “polysorbate 85” or “TWEEN® 85”), polyoxyethylene sorbitan monopalmitate (also known as “polysorbate 40” or “TWEEN® 40”), polyoxyethylene sorbitan monostearate (also known as “polysorbate 60” or “TWEEN® 60”), or a combination thereof.

The term “transition metal” in the context of the present disclosure refers to a metallic element selected from the groups Ib, IIb, IIIa (including the lanthanides), IVa, Va VIa, VIIa and VIII of the Periodic Table of the Elements that have an incomplete inner electron shell and may serve as transitional links between the most and the least electropositive in a series of elements. In other words, the term “transition metal” refers to an element whose atomic number lies between 21 and 30, between 39 and 48, between 57 and 80, or between 89 and 112.

The term “nucleic acid” refers to a polymer of nucleotides (e.g., ribonucleotides and deoxyribonucleotides, both natural and non-natural), including DNA, RNA, and their subcategories, such as cDNA, mRNA, etc. A nucleic acid may be single-stranded or double-stranded and is generally contain 5′-3′ phosphodiester bonds, although in some cases, nucleotide analogs may have other linkages as well as linkers, spacers and labels available in the art. Nucleic acids may include naturally occurring bases (adenosine, guanosine, cytosine, uracil and thymidine) as well as non-natural bases, which may have a particular function, e.g., increasing the stability of the nucleic acid duplex, inhibiting enzyme digestion or blocking primer extension or strand polymerization.

As used herein, the term “barcode” refers to a nucleotide sequence conferring identity to a molecule or a group of molecules sharing a common property or origin. A barcode may confer a unique identity to an individual molecule (and its copies); such a barcode is a unique ID (UID) or unique molecular identifier (UMI). A barcode may confer an identity to an entire population of molecules (and their copies) coming from the same source (e.g., a sample); such a barcode is a multiplex ID (MID) or sample ID (SID). Barcodes can be of sufficient length and comprise sequences that are sufficiently different to allow the identification of samples based on barcodes with which they are associated. For example, a barcode may have 4 to 40 nucleotides in length, 5 to 36 nucleotide in length, or 6 to 30 nucleotide in length. Each barcode in a barcode set has a unique nucleotide sequence, i.e., having a sequence differing from every other barcode in the barcode set. In some embodiments, the methods of the disclosure further comprise identifying the sample from which a target polynucleotide is derived based on the barcode sequence to which the target polynucleotide is joined. Barcode technologies are known in the art; see, for example, Winzeler et al., Science (1999), 285: 901; Kumar et al., Nature Rev. (2001), 2: 302; Giaever et al., Proc. Natl. Acad. Sci. USA (2004), 101: 793; Eason et al., Proc. Natl. Acad. Sci. USA (2004), 101: 11046; and Justus M. Kebschull et al., Nature Methods (2018), 15: 871. These publications are incorporated herein by reference in their entireties.

The term “primer” as used herein refers to an oligonucleotide, generally with a free 3′ hydroxyl group, that is capable of hybridizing or annealing with a template (such as a specific polynucleotide, target DNA, target RNA, and etc.) and promoting the polymerization of a polynucleotide complementary to the template. In addition to the hybridizing sequence that recognizes and binds to the template, a primer may further comprise a non-hybridizing sequence that constitutes a tail of the primer. For example, the barcoded RT primer of the present disclosure comprises a hybridizing sequence that recognizes and binds to target sequence of mRNA (e.g., polyadenylation (polyA) sequence of mRNA), and a barcode sequence linked to the 5′ or 3′ end (preferably 5′ end) of the hybridizing sequence. As could be appreciated, a primer can hybridize to a target sequence even though its sequences are not fully complementary to the target sequence.

As used herein, the term “adapter” refers to a nucleotide sequence that may be added to another sequence so as to import additional properties to that sequence. An adapter can be single- or double-stranded, or may have both a single-stranded portion and a double-stranded portion.

The term “amplifying” or “amplify” a specific nucleic acid refers to a procedure wherein multiple copies of the nucleic acid of interest are generated, for example, in the form of DNA copies. The methods and protocols for amplifying nucleic acids are known in the art, such as PCR and qPCR.

As used herein, the term “reverse transcription” refers to a reaction in which an RNA template is reverse transcribed using a reverse transcriptase into a cDNA. A reverse transcription reaction usually includes, RNA template, reverse transcriptase, reaction buffer (e.g., Tris or Tris-HCl), salt, primers (e.g., oligo(dT) primers or random primers), deoxynucleoside triphosphates (dNTPs), reducing agent (optionally; e.g., DTT) and RNase inhibitor (optionally). Depending upon the context, the mixture can be either a complete or incomplete reverse transcription reaction mixture.

### II. Description of the Invention

The present disclosure is based on the following discoveries: (1) the combination of Cu²⁺ ions and a suitable chelator (i.e., citrate or NTA) is capable of protecting RNA from degradation without causing undesirable protein aggregation in nuclease-rich sample; (2) Mg²⁺-containing methanol may serve as an efficient clumping-free nuclear stabilizer; and (3) Co²⁺ or Mn²⁺ ions are useful in tailing single-stranded DNA (ssDNA) or double-stranded DNA (dsDNA) with a recessed end. Based on these discoveries, the present disclosure provides a method of quantitatively analyzing and/or determining the expression profiles of single-cell RNA (scRNA) of a biological sample via isolating and stabilizing single nuclei from the cells of the biological sample, followed by adding a tag tail (e.g., barcode sequence) to each single nucleus, and then quantitatively analyzing and/or determining the expression profiles in accordance with the tag tail.

### (i) Methods of Inhibiting Nuclease Activity

The first aspect of the present disclosure is directed to a method of inhibiting the activity of a nuclease in a biological sample. According to certain embodiments of the present disclosure, the method comprises mixing the biological sample with a metal ion-chelator complex so as to inhibit the activity of the nuclease in the biological sample.

Depending on intended purpose, the biological sample may be any sample containing nuclease; for example, a sample derived from an animal tissue (e.g., spleen tissue, liver tissue, pancreatic tissue, bone marrow, or the cells derived/isolated therefrom) or a plant tissue (e.g., maize, or the cells derived/isolated therefrom).

In one embodiment of the present disclosure, the nuclease is DNase. In another embodiment of the present disclosure, the nuclease is RNase.

According to some embodiments of the present disclosure, the chelator may be a tridentate chelator, a quasidentate chelator or a pentadentate chelator that respectively links or binds with three, four or five metal ions via forming coordinate bonds therewith. In these embodiments, the hexadentate chelator (i.e., a chelator capable of linking or binding with six metal ions, such as EDTA) complexing with the metal ion fails to provide the inhibitory effect on the nuclease activity. According to certain exemplary embodiments, the metal ion-chelator complex is Cu-Citrate, Cu-NTA, or Cu-IDA.

Preferably, the molar ratio of the metal ion and chelator in the complex is ranges from 1 to 10; for example, the molar ratio of the metal ion and chelator in the complex may be 1:1, 2:1, 3:1, 4:1, 5:1, 6:1, 7:1, 8:1, 9:1, or 10:1. More preferably, the molar ratio of the metal ion and chelator in the complex equals to 1, i.e., the molar ratio of the metal ion and chelator in the complex is 1:1.

According to some embodiments of the present disclosure, the biological sample and metal ion-chelator is mixed in a buffer (e.g., phosphate buffered saline (PBS), Tris-HCl, or N-2-hydroxyethylpiperazine-N-2-ethane sulfonic acid (HEPES)), in which the metal ion-chelator is present in the buffer at a concentration of 2-100 mM (e.g., 2, 3, 4, 5, 6, 7, 8, 9, 10, 15, 20, 25, 30, 35, 40, 45, 50, 55, 60, 65, 70, 75, 80, 85, 90, 95, or 100 mM). In some embodiments, the buffer contains 10-50 mM of Cu-Citrate or Cu-NTA to suppress the nuclease activity. According to one specific example, the buffer for suppressing the nuclease activity contains 50 mM of Cu-Citrate. According to another specific example, the buffer contains 10 mM Cu-NTA to achieve the nuclease suppression purpose.

(ii) Methods of Isolating Nuclei from Cells

The second aspect of the present disclosure is directed to a method of isolating nuclei from cells in a biological sample by using the metal ion-chelator complex as described in section (i) of the present disclosure. The method comprises,
(a) mechanically disrupting the cells in the biological sample;
(b) mixing the product of step (a) with a lysis buffer in the presence of the present metal ion-chelator complex so as to release the nuclei from the cells; and
(c) separating the nuclei from the product of step (b) thereby producing the isolated nuclei.

According to various embodiments of the present disclosure, the biological sample may be a sample derived from an animal tissue (e.g., spleen tissue, liver tissue, pancreatic tissue, bone marrow, or the cells derived/isolated therefrom) or a plant tissue (e.g., maize, or the cells derived/isolated therefrom).

In step (a), the biological sample is physically disrupted (e.g., mechanically gridding) to release cell components therefrom. Examples of the physical disruption known to a skilled artisan in the art include, but are not limited to, bead method (also known as “bead beating”, in which small beads are mixed with the biological sample, followed by rapidly agitating the mixture that generates strong shear surrounding the cells of the biological sample and then pulls them apart), sonication (i.e., the use of ultrasonic homogenizer to induce vibration that triggers cavitation for shearing cells), grinding (i.e., creating friction by sandwiching the biological sample between two hard surfaces that slide against each other to achieve cell disruption), freezing (e.g., the use of liquid nitrogen, or freezing and thawing cycles that disrupt cells through ice crystal formation), and a combination thereof. According to some exemplary embodiments of the present disclosure, the biological sample is frozen by liquid nitrogen followed by aluminum plate grinding.

In step (b), the product of step (a) (i.e., the product after physically disruption) is mixed with a lysis buffer to completely release the nuclei from the cells. As known in the art, the lysis buffer is a buffer solution for cell breakage that usually contains a buffering salt (e.g., Tris-HCl, or HEPES) and ionic salts (e.g., NaCl, CaCl₂, and/or MgCl₂) to regulate the pH and osmolality of the lysate, and one or more detergents (e.g., TRITON® X-100, and/or sodium dodecyl sulfate (SDS)) to destroy membrane structures. According to some embodiments of the present disclosure, the lysis buffer contains 0.1-3% nonionic detergent. In one example of the present disclosure, the lysis buffer contains polysorbate 20 (TWEEN® 20) and nonyl phenoxypolyethoxylethanol (NP-40) as the detergent for the breaking purpose. Preferably, the lysis buffer comprises 0.01-1% (v/v) polysorbate 20 and 0.01-1% (v/v) nonyl phenoxypolyethoxylethanol that disrupts the plasma membrane, while keeps the nuclear membrane and/or component intact. Alternatively, the lysis buffer comprises 0.01-1% (v/v) polysorbate 20 and 0.01-1% (v/v) TRITON® X-100 or CA-630 for disrupting the plasma membrane while keeping the nuclear membrane and/or component intact.

Preferably, the lysis buffer further contains Cu-Citrate (IUPAC Name: dicopper; 2-hydroxypropane-1,2,3-tricarboxylic acid; also known as “cuprocitrol”; an ionic compound of copper and citric acid) or Cu-NTA (IUPAC Name: copper; 2-[bis(carboxylatomethyl)amino]acetate;hydron; also known as “copper-nitrilotriacetate; a complex formed by coupling copper ions and NTA) as a RNase inhibitor. According to some embodiments, the lysis buffer contains 2-100 mM of Cu-Citrate or Cu-NTA to inhibit the activity of RNase. In some embodiments, the lysis buffer contains 10-50 mM of Cu-Citrate or Cu-NTA to suppress the activity of RNase. According to one specific example, the lysis buffer for suppressing the RNase activity contains 50 mM of Cu-Citrate. According to another specific example, the lysis buffer contains 10 mM Cu-NTA to achieve the RNase suppression purpose.

In step (c), the nuclei are separated from the product of step (b). Depending on intended purpose, the nuclei separation/isolation may be carried out by FACS sorting, density gradient by using iodixanol or a modified sucrose gradient, or any commercial kits for nuclei isolation. The methods and procedures for nuclei separation/isolation are known by a person having ordinary skill in the art; hence, the detailed description thereof is omitted herein for the sake of brevity. According to some exemplary embodiments, the nuclei are separation/isolation by iodixanol gradient. In these embodiments, the product of step (b) is layered over a buffer containing iodixanol (at a low concentration; e.g., 30%) and Cu-Citrate, with iodixanol (at a high concentration; e.g., 60%) at the bottom; after centrifugation, the nuclei are collected from the interface of two layers of iodixanol (e.g., the interface of 30% iodixanol and 60% iodixanol).

Optionally, the method further comprises fixing the nuclei separated/isolated from step (c) with an anti-clumping agent (step (d)). As its name implies, the anti-clumping agent may prevent the nuclei from aggregation and clumping. According to the embodiments of the present disclosure, the anti-clumping agent is Mg²⁺-containing methanol. According to some embodiments, the methanol contains MgCl₂, i.e., methanol/MgCl₂. In certain exemplary embodiments, the Mg²⁺ ions are present in the methanol at a concentration of 5 mM.

(iii) Methods of Adding Deoxyribonucleotides to DNA

The third aspect of the present disclosure pertains to a method of adding deoxyribonucleotides to the 3′ end of DNA thereby extending or tailing the DNA. The method comprises,
(a) mixing the DNA with a reaction buffer comprising a terminal transferase, the deoxyribonucleotides, and a transition metal ion, wherein the reaction buffer is free of ions other than the transition metal ion (e.g., free of K⁺ ions, Mg²⁺ ions and Na⁺ ions); and
(b) incubating the mixture of step (a) at 37° C. for 30-120 minutes thereby producing the DNA having the deoxyribonucleotides added to its 3′-end.

According to various embodiments of the present disclosure, the DNA may be a ssDNA or a dsDNA. In one embodiment, the DNA is a cDNA. In another embodiment, the DNA is a dsDNA with a 3′ recessed (5′ overhang) end.

In step (a) the DNA is mixed with a reaction buffer containing a terminal transferase (an enzyme known to catalyze the addition of nucleotide to the 3′ hydroxyl terminus of DNA), and the deoxyribonucleotides (e.g., deoxyadenosine triphosphates (dATPs), deoxycytidine triphosphates (dCTPs), deoxythymidine triphosphates (dTTPs), and/or deoxyuridine triphosphates (dUTPs)). According to some exemplary embodiments, the deoxyribonucleotides are dATPs, in which the concentration of the dATPs in the reaction buffer ranges from 0.5 mM to 10 mM. In one specific example, the concentration of the dATPs in the reaction buffer is 2 mM.

According to some embodiments of the present disclosure, the terminal transferase is TdT. In these embodiments, the reaction buffer is TdT buffer (Tris-acetate).

The reaction buffer of the present method is characterized by having Co²⁺ or Mn²⁺ ions only, without containing other ions (including K⁺, Mg²⁺ and Na⁺ ions), i.e., only Co²⁺ or Mn²⁺ ions are present in the terminal transferase reaction.

In step (b), the mixture of DNA, terminal transferase and deoxyribonucleotides is incubated at about 37° C. for a period of time (e.g., 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 minutes) so that the deoxyribonucleotides are incorporated into the 3′ end of the DNA. Preferably, the mixture is incubated at about 37° C. for 60-120 minutes.

According to certain embodiments of the present disclosure, the DNA is mixed with TdT and dATPs in step (a), followed by incubation at 37° C. for 60 minutes so as to add a homopolymeric tail (polyadenylation (polyA) sequence) to the 3′ end of DNA.

(iv) Methods of Analyzing mRNA Profiles of Biological Sample

The fourth aspect of the present disclosure provides a method of analyzing the mRNA profiles of cells in a biological sample. The method comprises,
(a) mechanically disrupting the cells in the biological sample;
(b) mixing the product of step (a) with a lysis buffer in the presence of the present metal ion-chelator complex so as to release the nuclei from the cells;
(c) separating the nuclei from the product of step (b);
(d) fixing the nuclei of step (c) with a solution comprising methanol and magnesium ions (Mg²⁺);
(e) mixing the product of step (d) with a RT buffer comprising a reverse transcriptase, dNTPs, and a plurality of barcoded RT primers;
(f) subjecting the mixture of step (e) to RT reaction thereby producing a plurality of cDNAs, each of which comprises a first unique barcode sequence;
(g) mixing the plurality of cDNAs of step (f) with a reaction buffer comprising a terminal transferase (e.g., TdT), dATPs, and Co²⁺ or Mn²⁺ ions, wherein the reaction buffer is free of K⁺, Mg²⁺ and Na⁺ ions;
(h) incubating the mixture of step (g) at 37° C. for a period of time thereby producing a plurality of poly-adenylated cDNAs (i.e., cDNAs having a polyA tail);
(i) producing a plurality of double-stranded cDNAs via subjecting the product of step (h) to PCR;
(j) sequencing the product of step (i) so as to determine the number of each unique barcode sequence; and
(k) determining the mRNA profiles based on the result of step (j).

The steps (a)-(d) of the present method are quite similar to those of the isolation method as described in section (i) of the present disclosure. Therefore, the detailed description thereof is omitted herein for the sake of brevity.

In steps (e) and (f), a plurality of cDNAs, each of which comprises a unique tag, are produced by the single nuclei isolated from step (d). Specifically, the single nuclei are respectively mixed with RT reagents (step (e)), followed by subjecting the mixture to RT reaction (step (f)). As known in the art, the RT reagents provide the necessary components for reversing transcribing mRNA into cDNA; to this purpose, the RT reagent usually include reverse transcriptase, dNTPs, RT buffer (e.g., Tris or Tris-HCl), and a plurality of RT primers. According to some embodiments of the present disclosure, each RT primer is a barcoded RT primer that in its structure comprises a target-complementary sequence and a barcode sequence. As the name implies, the target-complementary sequence comprises a nucleotide sequence complementary to and exhibits binding affinity towards a target sequence of the mRNAs in the isolated nuclei, for example, an oligo(dT) sequence recognizes and binds to the polyA tail of mRNAs. The barcode sequence may have at least 4 nucleotides (e.g., 4, 5, 6, 7, 8, 9, 10, 11, 12, 13, 14, 15, 16, 17, 18, 19, 20, or more nucleotides) in length. According to one exemplary embodiment, the barcode sequence of each RT primer has 4 nucleotides in length. According to another exemplary embodiment, the barcode sequence of each RT primer has 8 nucleotides in length. According to another exemplary embodiment, the barcode sequence of each RT primer has 16 nucleotides in length. In this case, the barcode sequence would be incorporated into the cDNA during its synthesis and thus may act as a tag to identify the cDNA. Based on the distinct tag, once the tagged cDNAs are pooled, the tag may be used to identify single cell from which each cDNA originated. Thus, each cDNA corresponds to a single cell.

The concentrations of RT reagents and the procedures performing RT are known by a person having ordinary skill in the art. For the sake of brevity, the detailed descriptions thereof are omitted herein.

Optionally, the tagged cDNAs of step (f) (i.e., the cDNAs having a first unique tag coupled to its 3′ end) are respectively modified to incorporate another tag (i.e., a second unique tag) thereby diversifying the tag labeling. According to some optional embodiments, the tagged cDNAs of step (f) are mixed with a DNA ligation buffer comprising a DNA ligase and a plurality of adapters, wherein each of the adapters is characterized by having a second unique tag sequence, i.e., a distinct barcode sequence differing from every other barcode in the adapter set (step (f-1)). The mixture is then incubated at 37° C. for a period of time (for example, 30-120 minutes) to incorporate the second unique barcode sequence to the 3′ end of the first unique barcode sequence (step (f-2)).

Depending on intended purpose, steps (f-1) and (f-2) are optionally repeated at least once, for example, 1, 2, 3, 4, or more times.

The steps (g) and (h) of the present method are quite similar to those of the method as described in section (ii) of the present disclosure, and further details are omitted herein for the sake of brevity.

In step (i), the poly-adenylated cDNAs (i.e., a first strand) produced in step (h) serves as a template for synthesizing its complementary strand (i.e., a second strand complementary to the first strand) via PCR. The steps and conditions for producing dsDNAs from cDNAs via PCR are known by the person having ordinary skill in the art; for the sake of brevity, further detailed descriptions are omitted herein. According to certain working examples of the present disclosure, the PCR is carried out by mixing the cDNAs of step (h) (serving as templates) with DNA polymerase, oligo(dT) primer, dNTPs (including dATPs, dTTPs, dCTPs and dGTPs) and reaction buffer (e.g., Tris-HCl). Depending on intended purposes, the reaction buffer may contain one or more salts (e.g., MgCl₂).

Optionally, the dsDNAs produced in step (i) are further subjected to PCR for amplifying the tagged poly-adenylated cDNAs, i.e., increasing the copy number of the tagged poly-adenylated cDNAs.

Optionally, the PCR product is purified via a suitable method to separate the tagged poly-adenylated cDNAs from PCR reagents (i.e., primers and dNTPs); for example, column purification, gel purification, ethanol precipitation, polyethylene glycol (PEG) precipitation, magnetic beads, and etc. According to some exemplary embodiments of the present disclosure, the PCR product is mixed with magnetic beads in the presence of a PEG/NaCl solution, followed by subjecting the mixture to a magnetic stand.

Still optionally, the PCR product is enzymatically digested (e.g., the use of transposases) or mechanically sheared (e.g., the use of ultrasound, focused acoustic shearing, hydrodynamic shearing or nebulization to create shear force) into DNA fragments for DNA sequencing.

In step (j), the nucleotide sequences of dsDNAs are determined by a sequencing assay, for example, next generation sequencing (NGS) or Sanger sequencing.

Then, in step (k), the RNA profiles are determined based on the sequencing result of step (J). Specifically, as described above, the barcode tag(s) is/are useful in identifying each cDNA, its corresponding mRNA, and the single cell from which the mRNA/cDNA originated. Thus, the expression level of each mRNA may be determined by the number of the unique barcode sequence corresponding thereto.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Materials and Methods

### Preparation of Nucleic Acids for Barcoding

A total of 26 nucleic acids were synthesized in the present disclosure. The nucleotide sequences of these nucleic acids were summarized in Table 1.

TABLE 1
Nucleotide sequences of specified nucleic acids

| | | SEQ ID | |
|---|---|---|---|
| Name | Nucleotide sequence | NO | Note |
| SSDNA | GTGGAAAGGACGAAACAC | 1 | |
| | CGGATGCTTCCTTTTAAA | | |
| | CAGGGTTTTAGAGCTAGA | | |
| | AATA | | |
| 3′ recessed | TACCCTACTACTCTCACCA | 2 | Annealed region underscored. |
| end | CCATCTCTACCACTTC | | |
| dsDNA1 | | | |
| 3′ recessed | GTGATGGTTAGTGAGGAA | 3 | Annealed region underscored. |
| end | GTGGTAGAGATGGT | | |
| dsDNA2 | | | |
| T16V | CTCACTAACCATCACTC- | 4 | NNNN: 4-base barcodes. |
| primer | NNNN-nnnn- | | nnnn: 4-base unique molecular |
| | TTTTTTTTTTTTTTTT | | identifiers (UMIs). |
| | | | Each N and n was independently |
| | | | selected from the group |
| | | | consisting of nucleotides A, T, C |
| | | | and G. |
| | | | 5′ end was phosphorylated. |
| N9 primer | CTCACTAACCATCACTC- | 5 | NNNN: 4-base barcodes. |
| | NNNN-nnnn-NNNNNNNNN | | NNNNNNNNN: 9-base |
| | | | barcodes. |
| | | | nnnn: 4-base unique molecular |
| | | | identifiers (UMIs). |
| | | | Each N and n was independently |
| | | | selected from the group |
| | | | consisting of nucleotides A, T, C |
| | | | and G. |
| | | | 5′ end was phosphorylated. |
| Barcoding | TACCCTACTACTCTCAC- | 6 | NNNNNNNN: 8-base barcodes; |
| adapter | NNNNNNNN- | | each N was independently |
| (BC1) | CACCATCTCTACCACTTC | | selected from the group |
| | | | consisting of nucleotides A, T, C |
| | | | and G. |
| | | | Annealed region with SEQ ID |
| | | | NO: 7 was marked in bold font. |
| | | | 5′ end was phosphorylated. |
| Splinter for | TTAGTGAGGAAGTGGTAG | 7 | Annealed region with SEQ ID |
| BC1-BC0 | AGATGGT | | NO: 6 was marked in bold font. |
| Barcoding | TATAGAATTCGCGGCCGCT | 8 | NNNNNNNN: 8-base barcodes; |
| adapter | CGCGATAGC-NNNNNNNN- | | each N was independently |
| (BC2) | ATCCTCCTACTCTCACCA | | selected from the group |
| | A | | consisting of nucleotides A, T, C |
| | | | and G. |
| | | | Annealed region with SEQ ID |
| | | | NO: 9 was marked in bold font. |
| Splinter for | GTAGGGTATTGGTGAGA | 9 | Annealed region with SEQ ID |
| BC2-BC1 | GTAGGAGGA | | NO: 8 was marked in bold font. |
| Barcoding | GACGCAGT-NNNN-TC | 10 | NNNN: 4-base barcodes; each N |
| adapter | TGCCAAAAGTCGAGT | | was independently selected from |
| (BC1.5) | TTC | | the group consisting of |
| | | | nucleotides A, T, C and G. |
| | | | Annealed region with SEQ ID |
| | | | NO: 11 was marked in bold font. |
| | | | 5′ end was phosphorylated. |
| Splinter for | GTAGGGTAGAAACTCGAC | 11 | Annealed region with SEQ ID |
| BC1.5-BC1 | TTTTGGCAGA | | NO: 10 was marked in bold font. |
| Splinter for | ACTGCGTCTTGGTGAGA | 12 | Annealed region with SEQ ID |
| BC2-BC1.5 | GTAGGAGGAT | | NO: 8 was marked in bold font. |
| sup T25V | TATAGAATTCGCGGCCGCT | 13 | phosphorothioate bond |
| | CGCGATTTTTTTTTTTTTT | | |
| | TTTTTTTTTTT*V | | |
| suppressive | TGTATAGAATTCGCGGCCG | 14 | |
| primer | CTCGCGAT | | |
| pTn5MErev | CTGTCTCTTATACACATCT | 15 | 5′ end was phosphorylated. |
| 15ME | TCGTCGGCAGCGTCAGAT | 16 | Annealed region with SEQ ID |
| | GTGTATAAGAGACAG | | NO: 15 was marked in bold font. |
| i5 | TCGTCGGCAGCGTC | 17 | |
| supAGC | T*G*T*A*TAGAATTCGCG | 18 | *: phosphorothioate bond |
| | GCCGCTCGCGAT*A*G*C | | |
| P7supAGC | CAAGCAGAAGACGGCATA | 19 | *: phosphorothioate bond |
| | CGAGATGTATAGAATTCGC | | |
| | GGCCGCTCGCGATA*G*C | | |
| Nextera i5 | AATGATACGGCGACCACC | 20 | *: phosphorothioate bond |
| | GAGATCTACAC- | | N: 8-base barcodes for library |
| | NNNNNNNN- | | multiplexing; each N was |
| | TCGTCGGCAGCGT*C | | independently selected from the |
| | | | group consisting of nucleotides |
| | | | A, T, C and G |
| GAPDH F | ACAAGAGGAAGAGAGAG | 21 | For qPCR in Example 12 |
| | ACC | | |
| GAPDH R | CCTCTTCAAGGGGTCTAC | 22 | For qPCR in Example 12 |
| | AT | | |
| PSMB4 F | GGCTTTGAATGAAATACA | 23 | For qPCR in Example 12 |
| | GATG | | |
| PSMB4 R | AGAAGAGTCTATCTTTGA | 24 | For qPCR in Example 12 |
| | ACTAGC | | |
| genomic | GTAACCTCACTTTCCTGCT | 25 | For qPCR in Example 12 |
| DNA F | C | | |
| genomic | AGCCTGAAAATTACCCATC | 26 | For qPCR in Example 12 |
| DNA R | C | | |

### Example 1 Monovalent Cations and Mg²⁺ Utilized in all Current Reaction Systems are not Required for TdT Extension on ssDNA Substrates

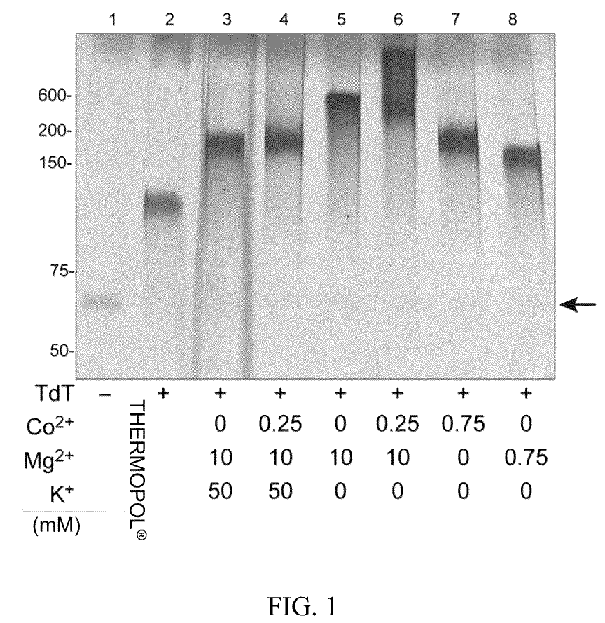

0.5 pmole of an ssDNA oligo (SEQ ID NO: 1) was incubated in 10 μL volumes of various compositions plus dATP (2 mM) and commercial TdT (0.25 μL) at 37° C., 45° C., 55° C., 65° C., 75° C. for 30, 5, 5, 5, 20 minutes, respectively. All the reaction was used for electrophoresis (FIG. 1).

With current conventional example, TdT relied on ThermoPol buffer, which contained the monovalent cation K⁺ that was detrimental to tailing (FIG. 1, lanes 3 & 4 vs. 5 & 6). Besides, the enzymic cofactor in this buffer was Mg², which was known suboptimal for TdT, particularly with blunt or recessed-end substrates(2), such as many cDNA ends found on mRNA. Consistent with the logic, a single-stranded substrate extended in ThermoPol or Mg²⁺-based buffer yielded shorter tails than that in a Co²⁺-containing buffer (FIG. 1, lanes 2 & 8 vs. 7, respectively) and Co²⁺ could replace Mg²⁺ for tailing the ssDNA (FIG. 1, lane 7 vs. 8).

Overall, these findings indicate that the cations commonly used in TdT reactions are detrimental for adding poly(dA) tails to ssDNA substrates, while Co²⁺ alone is sufficient for this purpose and achieves the highest efficiency compared to the other reaction systems.

### Example 2 the Novel Ion-Free Formulation Enabled TdT to Extend 3′ Recessed-End dsDNA Substrates with 100% Efficiency, a Result not Achievable with all Previous Methods

3′ recessed dsDNA was prepared by annealing 25 μM each of SEQ ID NO: 2 and SEQ ID NO: 3 in Tris-HCl pH 8 (25 mM), NaCl (12.5 mM), and EDTA (0.25 mM). 2.5 pmole of the annealed substrate was incubated in 20 μL of reaction mixtures containing PMSF (0.5 mM), dATP (2 mM), TRITON® X-100 (0.1%), Tris-acetate pH 7.9 (20 mM), and either Mg²⁺ or Co²⁺ in chloride salt form (M²⁺, 0.75 mM) as tailing cofactors, with (+) or without (−) of TdT (0.25 μL) at 37° C. for 60 minutes followed by 75° C. for 20 minutes. Subsequently, all the reactions were loaded for electrophoresis.

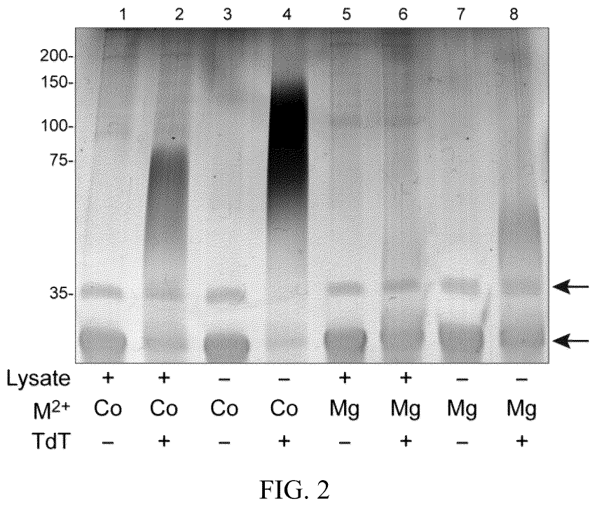

Here, it was found the substitution of Co²⁺ for Mg²⁺ to rescue suboptimal tailing was even more prominent with a recessed-end substrate, as evidenced by the nearly complete upshift of the substrate when Mg²⁺ was replaced by Co²⁺ (FIG. 2, lane 8 vs. 4).

The data distinguish the reaction system in this invention from all existing ones by its ability to achieve nearly 100% tailing of the most difficult type of dsDNA ends. Not only does the demonstration using the Mg²⁺ in the data here show this, but the inability to completely extend the 3′ recessed end was also evident in the past with different Co²⁺-containing formulations. This indicates that Co²⁺ alone was not the sufficient determining factor for complete tailing, but rather the absence of other inhibitory ions was crucial. Additionally, the efficient extension of both ssDNA (with accessible 3′ protruding ends, as shown in FIG. 1) and 3′ recessed-end dsDNA (with the most inaccessible ends, as shown in FIG. 2) indicates that the reaction system can comprehensively extend DNA ends in a manner not achievable with existing reaction systems.

### Example 3 Complete Tailing Required the Combination of Either Co²⁺ or Mn²⁺ with dATP, but not dGTP

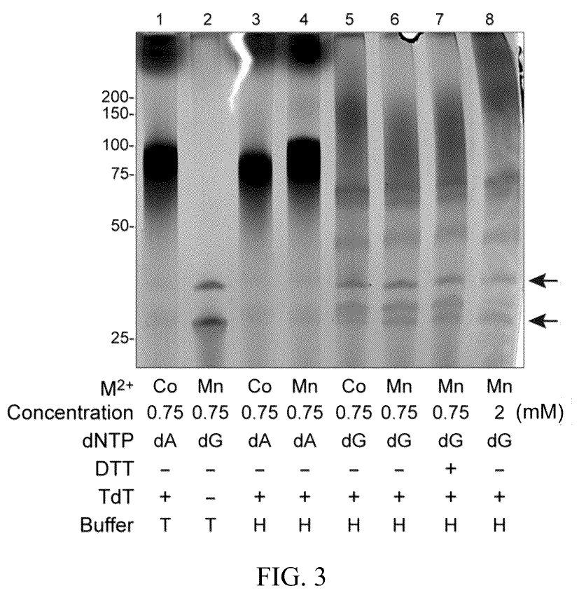

5 pmole of the 3′ recessed-end substrate as in Example 2 was incubated in 10 μL of reaction containing HEPES (H) or Tris-acetate (T) pH 7.9 (20 mM), dATP (dA) or dGTP (dG) (2 mM), CoCl₂ (Co) or MnCl₂ (Mn) (0.75 or 2 mM), DTT (0 or 0.2 mM), and TdT (0 or 0.25 μL) at 37° C. for 60 minutes followed by 75° C. for 20 minutes before loading half of the samples for electrophoresis (FIG. 3).

In addition to the optimal buffer based on dATP and Co²⁺, two other alternative ingredients, Mn²⁺ and dGTP, reported to improve tailing, were found either not to improve (FIG. 3, lanes 4 vs. 3) or even to be detrimental (FIG. 3, lanes 5, 6 vs. 3, 4). Besides, neither including dithiothreitol (DTT), a reducing agent (FIG. 3, lanes 7 vs. 6), nor replacing Tris-HCl with HEPES, an alternative buffer (FIG. 3, lanes 1 vs. 3), apparently enhanced the tailing.

The data demonstrate that the formulation in this invention is flexible with respect to the two types of transition metals (but not alkaline earth metals) and the pH control buffers used, such as HEPES and TRIS. The presence of the reducing agent DTT is optional and does not affect the results. However, the choice of dNTPs (dATP instead of dGTP) is crucial for achieving complete extension.

### Example 4 Complete Tailing Required a Sufficient Amount of TdT Relative to the Amount of Substrate

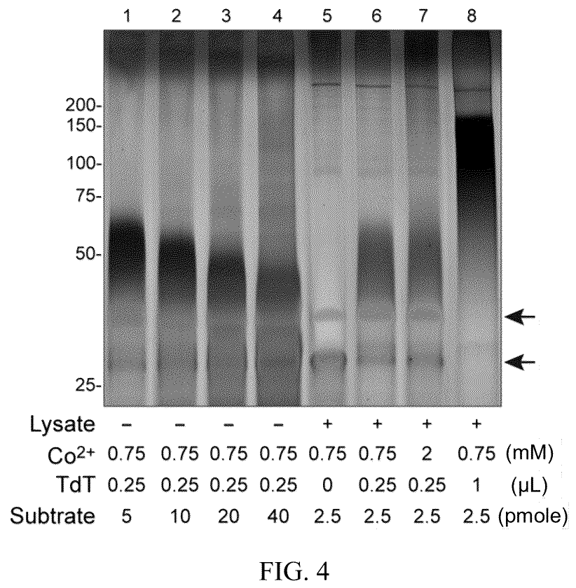

Various pmoles of the 3′ recessed-end substrate prepared as in Example 2 were incubated in 10 μL of reactions containing PMSF (0.5 mM), Tris-acetate pH 7.9 (20 mM), TRITON® X-100 (0.1%), dATP (2 mM), CoCl₂ (0.75 or 2 mM) and commercial TdT (0, 0.25 or 1 μL) with (+) or without (−) the 5000-cell lysate (Lysate) at 37° C. for 60 minutes followed 75° C. for 20 minutes before loading all of reactions for electrophoresis (FIG. 4).

With the finalized formulation (Tris-acetate pH 7.9 20 mM, dATP 2 mM, CoCl₂ 0.75 mM, TRITON® X-100 0.1%) and various amount of TdT (20 U/μl), 5 unites of TdT efficiently tailed 2.5 pmole of the recessed-end substrate (FIG. 2, lane 4). However, the tailing efficiency dropped significantly either when the substrate concentrations increased (FIG. 4, lanes 1-4) or lysates containing 5000-cell cDNA were included in the reaction (FIG. 2, lanes 2 vs. 4). This reduced tailing efficiency due to substrate quantity could be addressed by increasing the TdT concentration, but not by increasing the cofactor Co²⁺ (FIG. 4, lane 6 vs. 8 and 7, respectively).

In conclusion, this TdT reaction system based on optimizing ingredient types and concentrations enabled the complete tailing of recessed-end DNAs, a task that was impossible in the past. Additionally, the simple reaction formulation permitted direct continuation into procedures, such as PCR amplification, by simply chelating residual Co²⁺.

### Example 5 Cu²⁺ Inhibited Nuclease Activity in Pancreatic Lysate

The crude pancreatic lysate was prepared by lyzing 10 mg of murine pancreas in 400 μL of lysis buffer (HEPES pH 7.2 20 mM, NaCl 146 mM, CaCl₂ 1 mM, MgCl₂ 21 mM, TWEEN® 20 0.1%, NP-40 0.1%, and digitonin 0.01%). The lysis was performed by bead beating (10 seconds, 4500 rpm) in 2 ml plastic tubes containing 6 glass beads of 2.5-mm diameter. The lysate was subjected to centrifugation at 17,000 RCF for 1 minute before the cleared nuclease-laden supernatant was kept for the assays.

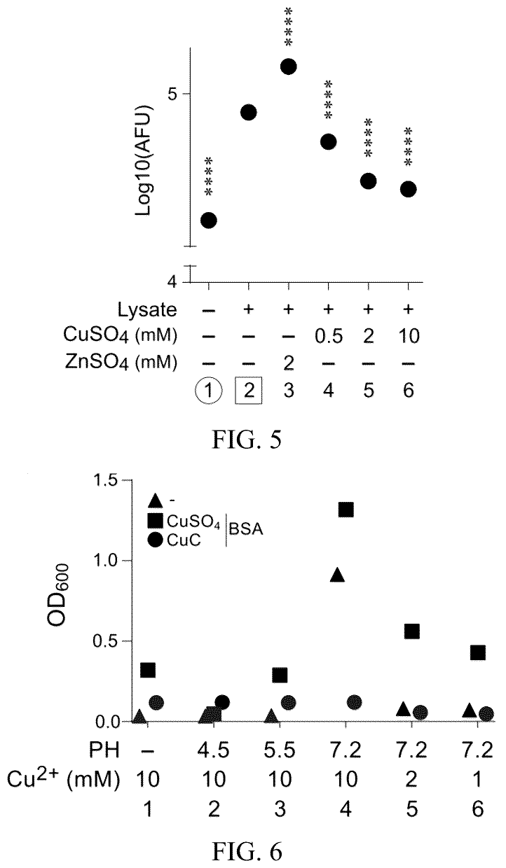

To detect the nuclease activity, an RNaseAlert™ oligo made of chimeric DNA-RNA-DNA with a green fluorescence molecule on one end and a quencher on the other (50 nM) was used in 25 μL final reaction volume. The reactions were conducted in the pancreatic lysis buffer with a final volume of 25 μL, containing the specified additives (pancreatic lyate, CuSO₄, or ZnSO₄) in FIG. 5. After incubating at ambient temperature for 30 minutes, the reactions were diluted with 150 μL of the same buffer, excluding substrates and the lysate. Fluorescence detection was performed in triplicate, with excitation at 490 nm and emission at 520 nm.

Here, it is evident that Cu²⁺ effectively diminished the nuclease activity in pancreatic lysate dependent on concentrations (FIG. 5).

### Example 6 Cu²⁺ Suffered from Precipitation Issues, which were Significantly Worsened in the Presence of Proteins Such as Albumin, Rendering it Unsuitable for Biological and Clinical Applications

A total of 180 μL of reactions per well were prepared at ambient temperature in a 96-well plate. These reactions contained various buffers (none or 100 mM) at specified pH levels, Cu²⁺ salts in either sulfate (no pH adjustment) or citrate form (at pH 4.5 or 7.2, as specified) at the indicated concentrations, and bovine serum albumin (10 g/L final concentration) in FIG. 6. The reactions were prepared in triplicate, photographed directly (FIG. 6, lower), and measured for optical density at 600 nm (FIG. 6, upper) to indicate precipitation.

The hydroxylated form of Cu²⁺ was only minimally soluble in water at neutral pH, even at concentrations as low as 1 mM (FIG. 6, lanes 4-6, triangle). Besides, Cu²⁺ led to severe protein aggregation at pH above 5.5, as evidenced by the co-incubation of this ion with bovine serum albumin (BSA), which caused precipitation both visually and as indicated by OD600 readings (FIG. 6, lanes 3-6, square). The precipitation makes Cu²⁺ alone unsuitable for use in biological and clinical applications because most samples contain proteins and are at neutral pH. However, precomplexing Cu²⁺ with citric acid, a chelator, resolved this issue at all pH values tested in the presence of BSA (FIG. 6, circle).

### Example 7 Chelated Cu²⁺ Remained Similarly Effective at Inhibiting Nuclease Activities as Compared to Unchelated Cu²⁺

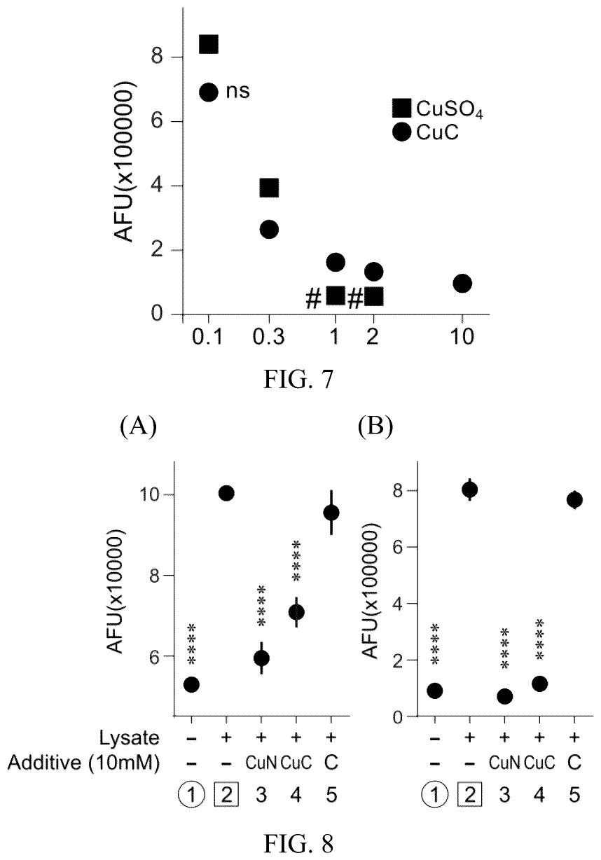

The RNaseAlert™ oligo (50 nM) was prepared in pancreatic lysis buffer with a final volume of 25 μL, containing the specified additives (pancreatic lysate, CuSO₄, or Cu²⁺-citric acid at various concentrations) in FIG. 7. After incubating at ambient temperature for 30 minutes, the reactions were diluted to 150 μL with the same buffer, excluding substrates and lysate, for fluorescence quantification in triplicate of 50 μL.

The experiment showed the chelated Cu²⁺ was still capable of blocking pancreatic nuclease activity in a dose-dependent manner, similar or even more potent to the unchelated form (FIG. 7, circles vs. squares). This indicates that chelators maintain the beneficial effects on nuclease inhibition while avoiding the problematic issue of precipitation, making the complex useful for biological systems.

### Example 8 the Chelator Itself Did not Inhibit Nucleases, but the Complexes Formed are Active Against Nucleases at Both Acidic and Neutral pH Levels

The RNaseAlert™ oligo (50 nM) was prepared in pancreatic lysis buffer, either HEPES-SO₄ at pH 4.5 or HEPES at pH 7.2, with a final volume of 25 μL, containing the specified additives (pancreatic lysate, Cu²⁺, or chelators) and pH levels (pH 4.5 or 7.2) in FIG. 8. After incubating at ambient temperature for 30 minutes, the reactions were diluted to 150 μL with the same buffer, excluding substrates and lysate, for fluorescence quantification in triplicate of 50 μL.

This experiment demonstrates that the nuclease-blocking activity was dependent on Cu²⁺ and not on the chelator alone (FIG. 8, pH 4.5 and 7.2, lanes 5 vs. 3 & 4). Further, the Cu²⁺-citric acid complex was active at both acidic and neutral pH (FIG. 8, pH 4.5 & 7.2, lanes 4 vs. 2).

### Example 9 the Cu²⁺-Chelator Complex was a Broad-Spectrum Nuclease Inhibitor Effective Against Both DNases and RNases

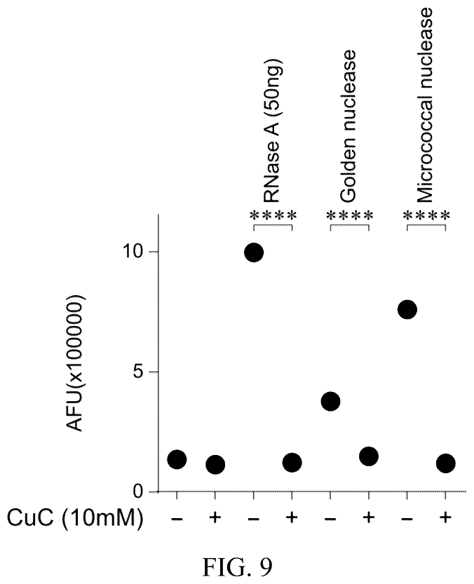

The RNaseAlert™ oligo (50 nM) was prepared in pancreatic lysis buffer with a final volume of 25 μL, containing the specified additives (pancreatic lysate, Cu²⁺-citric acid, or various nucleases) in FIG. 9. After incubating at ambient temperature for 10 minutes, the reactions were diluted to 150 μL with the same buffer, excluding substrates and lysate, for fluorescence quantification in triplicate of 50 μL.

The experiment demonstrates that the Cu²⁺-citric acid complex was active at both acidic and neutral pH (FIG. 9, pH 4.5 & 7.2, lanes 4 vs. 2) and was capable of blocking the activities of other nonselective DNA/RNA endonucleases, such as micrococcal nuclease and benzonase, in addition to RNase A (FIG. 9, lanes 4, 6, 8 vs. 3, 5, 7), indicating its broad-spectrum inhibition of different nuclease types, including DNases and RNases.

### Example 10 the Coordination Sites Occupied by the Chelators are Key in Determining the Effectiveness of Nuclease Inhibition

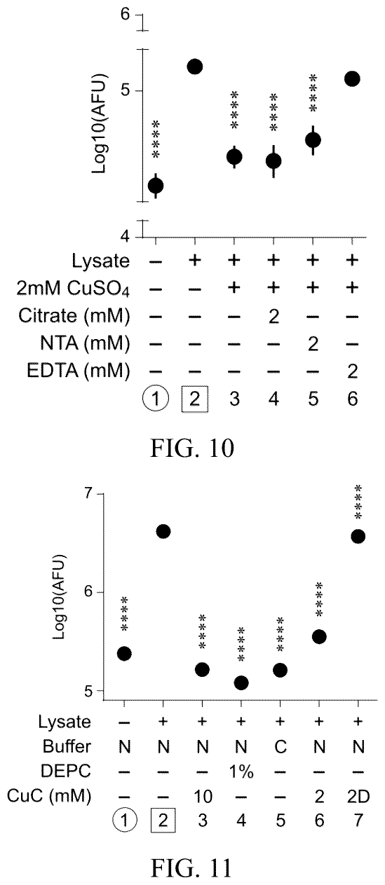

The RNaseAlert™ oligo (50 nM) was prepared in the pancreatic lysis buffer to a final volume of 25 μL, containing the specified additives (pancreatic lysate, 2 mM CuSO₄, or 2 mM of various chelators), as shown in FIG. 10. After incubating at ambient temperature for 30 minutes, the reactions were diluted to 150 μL with the same buffer, excluding substrates and lysate, for fluorescence quantification in triplicate of 50 μL.

The data indicated that the presence of Cu²⁺ with citric acid and nitrilotriacetic acid (NTA) still inhibited nuclease activity (FIGS. 10, lane 5; 3D, pH 4.5 & 7.2, lane 3). However, the presence of Cu²⁺ with ethylenediaminetetraacetic acid (EDTA) was ineffective in inhibiting nuclease activity (FIG. 10, lane 6). This differential partner requirement for Cu²⁺ highlights that the availability of free coordination sites (2/6 with citric acid and NTA, and 0/6 with EDTA) is crucial for achieving nuclease-blocking activity in the Cu²⁺-chelator complex, rather than the type of chelator itself.

### Example 11 the Cu²⁺-Chelator Complex Provided Nuclease Inhibition Comparable to the Current Best Methods, while Avoiding their Known Shortcomings

The RNaseAlert™ oligo (50 nM) was prepared in either the pancreatic lysis buffer (Buffer P) or a low-pH buffer (Buffer C, composed of 25 mM citrate at pH 3 and 250 mM sucrose) to a final volume of 25 μL. The preparation contained the specified additives (pancreatic lysate, 1% DEPC, and Cu²⁺-citric acid at specified concentrations) and is depicted in FIG. 11. After incubating at ambient temperature for 30 minutes, the reactions were diluted to 150 μL with the same buffer, excluding substrates and lysate, for fluorescence quantification in triplicate of 50 μL.

At the optimized concentration of 10 mM (FIG. 11, lane 3), this complex offered comparable RNase inhibition to the low-pH buffer (FIG. 11, lane 5), approaching the inhibition by DEPC (FIG. 11, lane 4). Finally, compared with the immediate inclusion of the Cu²⁺-chelator complex (FIG. 11, lane 6), the delayed addition of the Cu²⁺-chelator complex to the reaction showed minimal quenching of fluorescence (FIG. 11, lanes 7 vs. 2). This further supports the idea that the complex reduces fluorescence by preventing the cleavage of DNA/RNA that links the fluorophore to the quencher, rather than by directly quenching the fluorophore itself.

Overall, the metal-chelator complex demonstrated high nuclease-blocking efficiency without the known drawbacks of low pH-induced protein denaturation, nuclear distortion, or nucleic acid depurination, and DEPC-induced protein and nucleic acid modifications that hinder further applications, such as fixation.

### Example 12 the Cu²⁺-Chelator Complex Inhibited the Nuclease-Induced Degradation of Nuclear mRNA at Both Acidic and Neutral pH Levels

To mimic nuclear extraction by direct lysis of challenging organs, HEK293 cells were lysed in the presence of nuclease-rich pancreatic lysate, with or without the Cu²⁺-chelator complex: Thirty μL of pancreatic lysis buffer containing 50 mM HEPES at pH 4.5 or 7.2, 1× Halt proteinase inhibitor, 2 μL of pancreatic lysate, and either 0 mM (control) or 10 mM of Cu²⁺-citric acid complex from 250 mM stock solutions (at pH 4.5 and 7.2, respectively) were prepared on ice. 20,000 HEK293T cells in 2 μL of PBS were added to the lysis buffer, and the nuclei were released by rotating the cells in the fridge for 15 minutes. The nuclear pellets were obtained by centrifugation in a swing-bucket rotor at 1,000 g for 1 minute, washed twice with the same buffers excluding HALT, with the detergent in the wash buffer removed and replaced with 0.05% PVA. The washed nuclei were lysed with 15 μL of lysis buffer containing 0.2% TX-100 and 200 μg/μL proteinase K by vortexing at 1,200 rpm for 30 minutes at 37° C. The lysate was divided equally into 20 μL RT reactions, each containing RNA lysates, 0.2 μL of PMSF (50 mM in DMSO), 1× RT buffer (Tris-HCl pH 8.3, 50 mM; KCl, 75 mM; MgCl₂, 3 mM; DTT, 5 mM), dNTPs (final concentration 0.5 mM each), homemade rat RNase inhibitor (100 ng), PVA (0.05%), T23VN (1 μM), and either 0 (for genomic DNA control) or 100 ng of homemade reverse transcriptase. Reactions were incubated at 42° C. for 20 minutes, followed by 85° C. for 5 minutes. All of the cDNA was used as the input for triplicate qPCR reactions targeting housekeeping genes GAPDH (SEQ ID NO: 21 and SEQ ID NO: 22), PSMB4 (SEQ ID NO: 23 and SEQ ID NO: 24), and a segment of genomic DNA (SEQ ID NO: 25 and SEQ ID NO: 26), with each reaction volume being L.

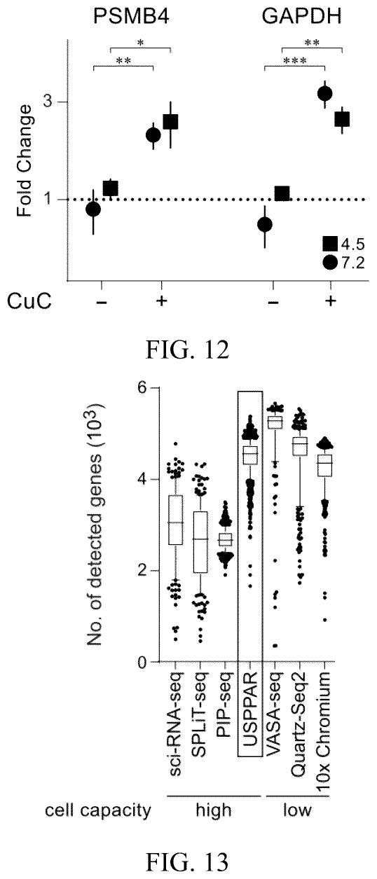

The Cu²⁺-citric acid complex was necessary to protect and retain nuclear RNA from complete degradation by pancreatic nuclease at both pH 4.5 and 7.2, when compared to nuclear samples prepared without this complex (FIG. 12, + and − CuC). This experiment demonstrates that the Cu²⁺-citric acid complex will be suitable for extracting nuclei for snRNA-seq, even with the most nuclease-laden samples.

### Example 13 scRNA-Seq Significantly Improved Gene-Detection Sensitivity Using the cDNA Tailing/Amplification System (Examples 1-4) Compared with Cells Prepared Using Similar RT and Barcoding Strategies

Cell Preparation for USPPAR (Unified System Having Split-Pool Barcoding with Optimal-Efficiency Polydeoxyadenylation for scRNA Detection)

The cultured cells were dissociated, followed by neutralization with an equal volume of PBSPVA (PBS containing 0.1% PVA), and wash again with the same volume of PBSPVA, with centrifugation in between at 3,000 RCF for 5 minutes. The pellets were resuspended with 20-40 μL of PBS, fixed by adding 10× volumes of methanol at −20° C., and permeated/fixed at −20° C. for at least 60 minutes before further use.

### First-Round Split-Pool Barcoding Based on RT

The required amounts of nuclei in methanol were added PVA to final 0.1% and underwent centrifugation at 1,000 g for 1 minute to remove methanol. The pellets were washed 2 more times with 200 μL of a low-salt buffer (Tris-HCl pH8 10 mM, KCl 1 mM, PVA 0.1%) containing recombinant rat RNase inhibitor (5 ng/μL). The nuclei were resuspended in the same buffer and aliquoted into a 96-well PCR plate for first-round barcoding (4,000 cells per 20 μL) using RT reaction. The RT reagent contained Tris-HCl pH8.3 50 mM, KCl 75 mM, MgCl₂ 3 mM, DTT 5 mM, dNTP 0.5 mM each, homemade rat RNase inhibitor 5 ng/μL, PVA 0.05%, PEG8K 7.5%, reverse transcriptase 5 ng/μL, barcoded T16V primer (SEQ ID NO: 4) 1 μM, barcoded N9 primer (SEQ ID NO: 5) 1 μM. The reaction was performed by rotating the whole plate at 42° C. for 40 minutes and stopped by adding 0.3 μL of EDTA 0.5 M to each well.

### Subsequent Rounds of Split-Pool Barcoding Based on Ligation

The stopped reactions were pooled into 2-4 microtubes, centrifuged, and washed twice with 200 μL of the low-salt buffer containing PVA (0.1%) and rat RNase inhibitor (0.5 ng/μL) by centrifugation at 1,000 RCF for 1 minute. The pellets were resuspended in the wash buffer and aliquoted for 20 μL of the following ligation reactions (Tris-HCl pH7.5 70 mM, MgCl₂ 10 mM, DTT 50 mM, ATP 1 mM, PVA 0.1%, rat RNase inhibitor 5 ng/μL, T4 DNA ligase 4 ng/μL, and barcoding adapter 1 μM prepared by annealing SEQ ID NO: 6 with SEQ ID NO: 7 for the first round, and SEQ ID NO: 8 with SEQ ID NO: 9 for the second round under the same conditions as described in Example 2).

The reaction was performed by rotating the whole plate at 37° C. for 40 minutes and stopped by adding 0.5 μL of EDTA 0.5 M to each well. Optionally, the ligation process may be repeated one, two or three times for diversifying the barcode sequences. After the last round of ligation, the stopped reactions were pooled and filtered through an 800-mesh-size nylon membrane. The cells were washed 1 time with 200 μL of the low-salt buffer containing PVA (0.1%) and rat RNase inhibitor (0.5 ng/μL) by centrifugation at 1,000 RCF for 1 minute. The pellets were resuspended with appropriate amounts of the wash buffer and aliquoted (about 10,000-20,000 cells in 3 μL per PCR tube) for long-term storage at −80° C.

Single-Tube Amplification of cDNA Using the Complete Tailing System Developed in Examples 2-4

The cells in 3 μL of low salt buffer were lysed by adding 8 μL of lysis buffer comprising TRITON® X-100 (0.275%) and proteinase K (275 g/ml). The lysis was conducted by vortexing at 1,500 rpm at 55° C. for 50 minutes. Each lysate was added with 2 μL of 10× TdT buffer (Tris-acetate pH7.9 200 mM, TRITON® X-100 1%), 2 μL of dATP (20 mM), 2 μL of CoCl₂ (7.5 mM), 0.2 μL of PMSF (50 mM in DMSO), commercial TdT (2 μL of 40 unites were sufficient for 10,000 cells) and water to achieve a final volume of 20 μL for polydeoxydenylation at 37° C. for 60 minutes and 42° C. for 10 minutes, followed by heat inactivation at 75° C. for 20 minutes. After the reaction, 1 μL of EDTA (16 mM) was added to make a trace-free completely extended cDNA.

The reactions were then added to a 58 μL mixture containing 16 μL of DNA polymerase buffer (5×), 1.6 μL of dNTP mixture (10 mM each), 8 μL of supT25V (SEQ ID NO: 13, 2 μM). The mixture was split into 2 equivalent 39 μL portions, heated at 94° C. for 1 minute, and held at 45° C. 1 μL of DNA polymerase was added to each reaction. Next, second-strand synthesis was performed with 54 cycles of 0.5° C. increase for 20 seconds each cycle, followed by 72° C. for 10 minutes and 4° C. indefinitely.

The reactions were pooled and added to an 80 μL mixture containing 16 μL of DNA polymerase buffer (5×), 1.6 μL of dNTP (10 mM each), 16 μL of suppressive primer (SEQ ID NO: 14, 5 μM), 8 μL of DMSO, and 2 μL of DNA polymerase. The reaction was heated to 98° C. for 1 minute, followed by 12 cycles of 98° C. for 10 seconds, 68° C. for 20 seconds, and 72° C. for 4 minutes. The PCR reaction was added with EDTA to a final concentration of 3 mM to stop the enzymatic activity.

### Purification of PCR Products

To purify the preamplified PCR product, the PCR reactions were supplemented with 1 μL of carboxylated paramagnetic beads and a PEG/NaCl solution composed of Tris-HCl pH8 (10 mM), PEG8000 (20%), NaCl (2.5M), EDTA (1 mM) and TWEEN® 20 (0.05%) to achieve a final PEG concentration of 7%. The mixture was incubated for 10 minutes and, subsequently, placed on a magnet stand. After removing the supernatant, the beads were washed twice with 500 μL of freshly prepared ethanol-water (80%) by inverting the tubes horizontally 10 times on the magnetic stand before removing the supernatant. The beads were left on the magnetic stand for 5 minutes to evaporate the remaining ethanol before elution using 8 μL of loTE (Tris-HCl pH8 10 mM, EDTA 0.1 mM).

A similar procedure was used to perform size selection on the tagmented cDNA except that a PEG concentration of 5.5% was used to remove high molecular-weight DNA first. The supernatant was removed from DNA-coated beads and supplemented with 1 μL of paramagnetic beads and additional PEG/NaCl solution to a final PEG concentration of 7% before incubation, washes, and finally elution using 8 μL of loTE containing TWEEN® 20 (0.1%).

### Tn5 Tagmentation of Preamplified DNA

The eluted DNA from the first-round PEG/NaCl purification was assayed using SYBR® Green dye with a calibration curve produced by plasmid DNA of known concentration. 50 ng of the preamplified DNA and 50 ng of the carrier plasmid were tagmented with 0.5 μL of Tn5 (2 μM) loaded with an annealed adapter (prepared by annealing SEQ ID NO: 15 with SEQ ID NO: 16 under the same conditions as described in Example 2) containing an i5 PCR handle in 10 μL of tagmentation buffer (Tris-HCl pH8.5 10 mM, MgCl₂ 5 mM, DMF 10%) at 55° C. for 30 minutes. The inactivation of Tn5 was performed by adding with 2.5 μL of a quench buffer (SDS 0.25%, EDTA 21 mM) and heating at 55° C. for additional 10 minutes. The optimal Tn5 amount was determined by pilot tagmentation with 100 ng of plasmid DNA, followed by inactivation and gel electrophoresis to target an intensity peak at around 800 bp. One L of the quenched reaction was used to determine the optimal cycle number in 10 μL of qPCR reaction composed of DNA polymerase buffer (1×), dNTP (0.2 mM each), i5 primer (SEQ ID NO: 17, 200 nM), supAGC primer (SEQ ID NO: 18, 200 nM), TRITON® X-100 (0.1%), SYBR® Green (1/30,000 v/v), ROX (500 nM), and 0.125 μL of DNA polymerase. The thermocycling includes initial denaturation of 98° C. for 1 minute, followed by 20 cycles of 98° C. for 10 seconds, 63° C. for 15 seconds, and 72° C. for 30 seconds. The cycle number that yielded half of maximum fluorescence was used to amplify the rest of the tagmented samples with identical PCR conditions, respectively replacing SYBR® Green, ROX, supAGC primer, and i5 primer with DMSO, water, P7supAGC (SEQ ID NO: 19), and Nextera i5 primer (SEQ ID NO: 20). Following adding EDTA (0.5 M) to achieve a final concentration of 3 mM, the PCR product underwent size selection using PEG/NaCl as described above.

### Results

These systemic optimizations were employed in USPPAR to collect single-cell transcriptomes from mixed human HEK293 and murine bone marrow MS5 cells with approximately 1,500 subsampled cells from a single preparation of 100,000 cells. First, the results revealed barcodes with clear species-specific enrichment in reads and minimal multiplets in a species-mixing experiment (data not shown). The low multiplet rates of 0% with 1,204 cells subsampled from a 100,000-cell input (FIG. 13A, 3-round barcoding) and 0.2% with 16,450 from approximately 400,000 cells were comparable to other high cell-throughput methods based on combinatorial barcoding, such as 1.3-1.7% with 10,000-13,000 from standard 100,000 cells using the SPLiT-seq kit, 3.3% with 39,400 cells using the PIP-seq v4 kit, and lower compared to the expected 6.4% with 16,000 cells using the commercial 10× Chromium HT channels. Notably, this low multiplet rate will not increase with the number of subsampled libraries in the same experiment because each library is endowed with another layer of barcode during library amplification.

Next, the sequencing readout was further benchmarked against reference datasets from several state-of-the-art platforms, including 10× Chromium (HEK293T and NIH/3T3 cells), Quartz-Seq2 (annotated HEK293 cells), VASA-seq (HEK293T and mouse embryonic stem cells, mESCs), sci-RNA-seq (HEK293T, HeLa S3, and NIH/3T3 cells), PIP-seq (HEK293T, and NIH/3T3 cells), and and SPLiT-seq (HEK293, HeLa S3, and NIH/3T3 cells). The datasets modeled together showed 4 well-defined clusters of HEK293 cells, HeLa S3, mouse embryonic stem cells (mESCs), and murine stromal cells, reflecting the input cell types of respective platforms. In addition to the co-clustering of cell types in the UMAP space, the pseudobulk reads from human cells were also segregated according to cell types. Beyond the distinctions by cell types, neighborhoods revealed technology-dependent differences: the correlations of outputs from sci-RNA-seq, SPLiT-seq, VASA-seq, and USPPAR were higher with each other. This was followed by those from 10× Chromium and Quartz-Seq2, and finally, that from PIP-seq. The similarity of USPPAR to sci-RNA-seq, SPLiT-seq, and VASA-seq was reflected by their common higher intronic percentages, a property that benefits the inference of cell-state transitions. To demonstrate the informativeness of the intronic reads, murine liver was used as the example because the mixture of HEK293 and MS5 cells was insufficient for cellular resolution. In this organ, the intronic counts alone could be independently modeled to yield distinct clusters reflecting proper cell types. This indicates that the reads aligned to intronic regions were informative and derived from transcripts, not from untranscribed genomic DNA.

To benchmark the detection sensitivity across technologies, the reads from HEK293 cells were subset from each platform to show UMIs and genes detected with the same read numbers per cell (FIG. 13, genes) and the cumulative numbers of detected genes from increasing randomly sampled cells. These assessments showed that the detection sensitivity based on USPPAR matched or approached those based on sorting or microfluidics (10× Chromium, VASA-seq, and Quartz-Seq2) and exceeded those of the other high-cell-throughput methods, SPLiT-seq, sci-RNA-seq and PIP-seq, all based on combinatorial barcoding. Regarding dropout probabilities by expression magnitude, USPPAR also showed low dropout rates similar to special equipment-based methods, with a higher number of genes passing the quality filter compared to the other three methods based on combinatorial barcoding (13,271 genes with USPPAR vs. 11,937, 11,302, and 10,433 with sci-RNA-seq, SPLiT-seq and PIP-seq, respectively).

Dependent on the complete DNA tailing reaction system in the first invention, USPPAR offered a higher gene detection efficiency compared to current high cell-throughput methods. It provided a scalable capacity of at least 100,000 to 400,000 cells, surpassing that of other high-sensitivity methods that depend on special equipment. Additionally, it also had low multiplet and dropout rates and good gene-expression correlation with other platforms.

Notably, compared with SPLiT-seq, a system that barcodes cells similarly to USPPAR but uses a template-switching method to tail cDNA for amplification, USPPAR detects a significantly greater number of genes. This further demonstrates the utility of the complete tailing system in this invention for DNA amplification in significantly enhancing gene detection.

### Example 14 the Optimized cDNA Tailing (Examples 1-4) and Nuclease Inhibition (Examples 5-12) Together Enabled snRNA-Seq to Capture Comprehensive Cell States from the Spleen in a Single Pot

### One-Pot Nuclear Extraction of Nuclease-Laden Murine Spleen

Mouse spleens were immediately frozen in liquid nitrogen until use. The tissue fragments were frozen in folded polyimide films in liquid nitrogen for at least 10 minutes, and then crushed using an aluminum block on a stainless iron plate submerged in liquid nitrogen to maintain the low temperature. The powder were immediately lysed with 1,000 μL of ice-cold lysis buffer (HEPES pH 4.5 20 mM, NaCl 146 mM, CaCl₂ 1 mM, MgCl₂ 21 mM) containing a detergent mixture (TWEEN® 20 0.1%, NP-40 0.1%, and digitonin 0.01%) and Cu²⁺-Citric acid (10 mM, diluted from a 250 mM stock solution at pH 4.5). The mixture was ground using plastic pestles in microcentrifuge tubes, and then filtered through an 800-mesh-size nylon membrane. Next, the filtrates were layered over 500 μL of the same lysis buffer containing Cu²⁺-Citric acid (10 mM), PVA (0.05%) to replace the detergent mixture, and iodixanol (30%), with 20 μL of iodixanol (60%) placed at the bottom as a cushion. After centrifugation at 2,000 g for 5 minutes, the nuclei were collected from the iodixanol 30%-60% interface, followed by washed twice with 200 μL of the wash buffer (HEPES pH 4.5 20 mM, NaCl 146 mM, CaCl₂ 1 mM, MgCl₂ 21 mM, PVA 0.05%, Cu²⁺-Citric acid 10 mM) in a column bearing hydrophilic PTFE membrane (3 m pore size). The nuclei were resuspended and fixed in 200 μL of wash buffer containing paraformaldehyde (0.125%) at 4° C. for 15 minutes. 10 μL Tris-HCl (pH 8, 1 M) were added to the nuclei to quench the paraformaldehyde. The nuclei were fixed with 10× volumes of methanol containing 5 mM final of MgCl₂ at −20° C. and remain at −20° C. until downstream usage. The downstream snRNA-seq procedure was identical to the scRNA-seq method described in Example 13, except that the nuclei were washed on the PTFE membrane during the split-pool barcoding process.

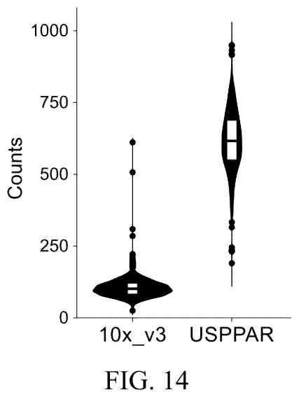

Given the combination of lysis buffer containing the Cu²⁺-chelator complex, dual fixation using PFA and methanol, and gentle cell washes on a filter membrane, mouse spleens, an RNase-rich bloody organ, were lysed in one pot and processed for snRNA-seq. The snRNA-seq approach rescued the loss of lymphocytic barcodes when using our original low-pH lysis buffer system. Additionally, standardizing the comparison by using the same number of barcodes from each Leiden cluster and maintaining a consistent read count per barcode (2,500) revealed an increased number of genes detected per barcode with the optimized snRNA-seq system, indicating its superior sensitivity. Further, a dataset from purified splenocytes obtained using the 10× commercial platform (Sample ID: 10k_Mouse_Splenocytes_5p_gemx, published on Apr. 16, 2024) was used for additional benchmarking. Our snRNA-seq results integrated well with the commercial dataset, revealing blood cell types with proportions comparable to those identified by the commercial 10× platform, which required selective enrichment of splenocytes (USPPAR vs. 10×_v3). Notably, our one-pot nuclear extraction revealed a more than tenfold increase in cells expressing mesenchyme- or endothelium-specific markers, Col1a1 and Cdh5, respectively. Based on the standardized comparison using barcodes belonging to the most abundant cell type, B cells, our system detected more genes than the commercial 10× platform when cells with the same number of subsampled reads were analyzed (FIG. 14, USPPAR vs. 10×_v3). Overall, the comparable percentages of blood cell types, increased coverage of mesenchymal cells, and higher gene detection sensitivity compared to the 10× platform demonstrate that our one-pot lysis system effectively handles challenging organs in a convenient manner.

In conclusion, unlike previous methods that required sorting incomplete subsets of individual cells from challenging organs such as the spleen, the strategy of nuclease inhibition in this invention permits the efficient and comprehensive capture of native cell states in the nuclease-rich organ. The improved sensitivity compared to the state-of-the-art 10× Chromium system was achieved through a unique combination of the broad-spectrum nuclease inhibitor and the complete cDNA tailing system.

### Example 15 the Combination of RNA Protection and DNA Amplification Also Enabled One-Pot Lysis of Plant Tissues, Improving Gene Detection Sensitivity in snRNA-Seq Compared to State-of-the-Art Commercial Platforms (Examples 1-12)

### One-Pot Nuclear Extraction of Nuclease-Laden Murine Spleen

The corn seeds were allowed to germinate on wet tissue paper for 4 days. The shoot fragments were frozen, crushed, and lysed in the same way as with the splenic fragments except a Ca²⁺-free lysis buffer (HEPES pH 4.5 20 mM, NaCl 146 mM, MgCl₂ 21 mM, and EGTA 0.5 mM) containing Cu²⁺-Citric acid (10 mM, diluted from a 250 mM stock solution at pH 4.5) and the detergent mixture was used for lysis. The lysates were further Dounce homogenized for 20 strokes to release nuclei, and then filtered through an 800-mesh-size nylon membrane. Next, the filtrates were layered over 500 μL of the same lysis buffer containing Cu²⁺-Citric acid (10 mM), PVA (0.05%) to replace the detergent mixture, and iodixanol (30%), with 20 μL of iodixanol (60%) placed at the bottom as a cushion. After centrifugation at 2,000 g for 5 minutes, the nuclei were collected from the iodixanol 30%-60% interface, followed by washed twice with 200 μL of the wash buffer (HEPES pH 4.5 20 mM, NaCl 146 mM, MgCl₂ 21 mM, EGTA 0.5 mM, PVA 0.05%, Cu²⁺-Citric acid 10 mM) by centrifugation at 250 g for 1 minute, resuspended with 20 μL of the same buffer containing Cu²⁺-Citric acid (10 mM), and fixed by adding 10× volumes of methanol containing 5 mM MgCl₂ at −20° C. for at least 30 minutes at −20° C. The downstream snRNA-seq procedure was identical to the scRNA-seq method described in Example 13, except that the nuclei were washed by centrifugation at 250 g for 1 minute to reduce the carryover of large organelles during the split-pool barcoding.

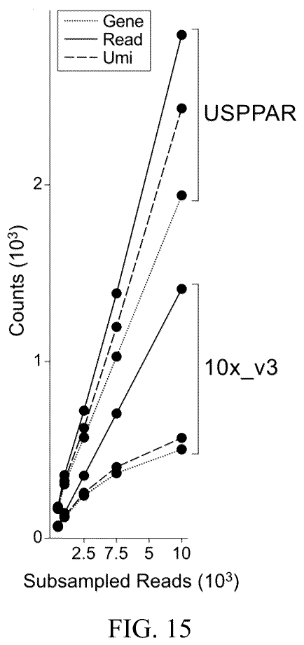

Compared to a reference nuclear dataset prepared using the 10× platform, nuclei prepared with USPPAR detected more UMIs and genes across all subsampled read numbers per barcode (FIG. 15, USPPAR vs. 10×_v3). Besides, multiplets were present at a very low level (0.2%) when nuclei from three different species were prepared simultaneously. Furthermore, the shoot dataset could be modeled and co-embedded with the reference (USPPAR vs. 10×_v3; data not shown). The inferred Leiden assignments remained well clustered when transferred to the 2D UMAP generated from the newly modeled and embedded using the USPPAR dataset alone. Finally, the informativeness of the data was further supported by the matched expression of markers between the reference and USPPAR datasets, such as Leiden3 with mesophyll marker Zm00001eb158810, Leiden11 with bundle-sheath marker Zm00001eb033390, and Leiden14 with protoxylem marker Zm00001eb076470.

It will be understood that the above description of embodiments is given by way of example only and that various modifications may be made by those with ordinary skill in the art. The above specification provides a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method of inhibiting the activity of a nuclease in a biological sample, comprising mixing the biological sample with a metal ion-chelator complex so as to inhibit the activity of the nuclease in the biological sample.

2. The method of claim 1, wherein the metal ion-chelator complex is copper citrate (Cu-Citrate), copper nitrilotriacetic acid (Cu-NTA), or copper iminodiacetic acid (Cu-IDA).

3. The method of claim 1, wherein the molar ratio of the metal ion and chelator in the complex ranges from 1 to 10.

4. The method of claim 3, wherein the molar ratio of the metal ion and chelator in the complex equals to 1.

5. A method of isolating nuclei from cells in a biological sample, comprising,
(a) mechanically disrupting the cells in the biological sample;
(b) mixing the product of step (a) with a lysis buffer in the presence of a metal ion-chelator complex so as to release the nuclei from the cells; and
(c) separating the nuclei from the product of step (b) thereby producing the isolated nuclei.

6. The method of claim 5, wherein in step (c), the nuclei are separated by steps of,
(c-1) subjecting the product of step (b) to density-gradient centrifugation in the presence of the metal ion-chelator complex; and
(c-2) collecting the fraction containing the nuclei from the product of step (c-1).

7. The method of claim 5, wherein the metal ion-chelator complex is copper citrate (Cu-Citrate), copper nitrilotriacetic acid (Cu-NTA), or copper iminodiacetic acid (Cu-IDA).

8. The method of claim 5, wherein the molar ratio of the metal ion and chelator in the complex ranges from 1 to 10.

9. The method of claim 8, wherein the molar ratio of the metal ion and chelator in the complex equals to 1.

10. The method of claim 5, wherein the lysis buffer of step (b) comprises 0.1-3% (v/v) nonionic detergent.

11. The method of claim 5, wherein the metal ion-chelator complex is present in the lysis buffer at a concentration of 2-100 mM.

12. The method of claim 5, further comprising
(d) fixing the nuclei of step (c) with a solution comprising methanol and magnesium ions (Mg²⁺).

13. The method of claim 12, wherein the Mg²⁺ ions are present in the solution at a concentration of 5 mM.

14. A method of adding a deoxyribonucleotide to the 3′ end of a deoxyribonucleic acid (DNA), comprising
(a) mixing the DNA with a reaction buffer comprising a terminal deoxynucleotidyl transferase (TdT), the deoxyribonucleotide, and a transition metal ion, wherein the reaction buffer is free of ions other than the transition metal ion; and
(b) incubating the mixture of step (a) at 37° C. for 30-120 minutes thereby producing the DNA having the deoxyribonucleotide added to its 3′-end.

15. The method of claim 14, wherein the transition metal ion is cobaltous ion (Co²⁺) or manganous ion (Mn²⁺).

16. The method of claim 14, wherein the DNA is a complementary deoxyribonucleic acid (cDNA).

17. The method of claim 14, wherein the DNA is a double-stranded DNA with a 3′ recessed end.

18. The method of claim 14, wherein the deoxyribonucleotide is deoxyadenosine triphosphate (dATP), deoxycytidine triphosphate (dCTP), deoxythymidine triphosphate (dTTP), or deoxyuridine triphosphate (dUTP).

19. The method of claim 18, wherein the deoxyribonucleotide is the dATP, and the concentration of the dATP in the reaction buffer ranges from 0.5 mM to 10 mM.

20. The method of claim 19, wherein the concentration of the dATP in the reaction buffer is 2 mM.


SEQUENCE LISTING (26 sequences)

SEQ ID NO: 1 - DNA, 58 bp, synthetic construct
  source 1..58: mol_type = other DNA
gtggaaaggacgaaacaccggatgcttccttttaaacagggttttagagctagaaata

SEQ ID NO: 2 - DNA, 35 bp, synthetic construct
  source 1..35: mol_type = other DNA
taccctactactctcaccaccatctctaccacttc

SEQ ID NO: 3 - DNA, 32 bp, synthetic construct
  source 1..32: mol_type = other DNA
gtgatggttagtgaggaagtggtagagatggt

SEQ ID NO: 4 - DNA, 41 bp, synthetic construct
  source 1..41: mol_type = other DNA
ctcactaaccatcactcnnnnnnnntttttttttttttttt

SEQ ID NO: 5 - DNA, 34 bp, synthetic construct
  source 1..34: mol_type = other DNA
ctcactaaccatcactcnnnnnnnnnnnnnnnnn

SEQ ID NO: 6 - DNA, 43 bp, synthetic construct
  source 1..43: mol_type = other DNA
taccctactactctcacnnnnnnnncaccatctctaccacttc

SEQ ID NO: 7 - DNA, 25 bp, synthetic construct
  source 1..25: mol_type = other DNA
ttagtgaggaagtggtagagatggt

SEQ ID NO: 8 - DNA, 55 bp, synthetic construct
  source 1..55: mol_type = other DNA
tatagaattcgcggccgctcgcgatagcnnnnnnnnatcctcctactctcaccaa

SEQ ID NO: 9 - DNA, 26 bp, synthetic construct
  source 1..26: mol_type = other DNA
gtagggtattggtgagagtaggagga

SEQ ID NO: 10 - DNA, 32 bp, synthetic construct
  source 1..32: mol_type = other DNA
gacgcagtnnnntctgccaaaagtcgagtttc

SEQ ID NO: 11 - DNA, 28 bp, synthetic construct
  source 1..28: mol_type = other DNA
gtagggtagaaactcgacttttggcaga

SEQ ID NO: 12 - DNA, 27 bp, synthetic construct
  source 1..27: mol_type = other DNA
actgcgtcttggtgagagtaggaggat

SEQ ID NO: 13 - DNA, 50 bp, synthetic construct
  source 1..50: mol_type = other DNA
tatagaattcgcggccgctcgcgatttttttttttttttttttttttttv

SEQ ID NO: 14 - DNA, 27 bp, synthetic construct
  source 1..27: mol_type = other DNA
tgtatagaattcgcggccgctcgcgat

SEQ ID NO: 15 - DNA, 19 bp, synthetic construct
  source 1..19: mol_type = other DNA
ctgtctcttatacacatct

SEQ ID NO: 16 - DNA, 33 bp, synthetic construct
  source 1..33: mol_type = other DNA
tcgtcggcagcgtcagatgtgtataagagacag

SEQ ID NO: 17 - DNA, 14 bp, synthetic construct
  source 1..14: mol_type = other DNA
tcgtcggcagcgtc

SEQ ID NO: 18 - DNA, 30 bp, synthetic construct
  source 1..30: mol_type = other DNA
tgtatagaattcgcggccgctcgcgatagc

SEQ ID NO: 19 - DNA, 53 bp, synthetic construct
  source 1..53: mol_type = other DNA
caagcagaagacggcatacgagatgtatagaattcgcggccgctcgcgatagc

SEQ ID NO: 20 - DNA, 51 bp, synthetic construct
  source 1..51: mol_type = other DNA
aatgatacggcgaccaccgagatctacacnnnnnnnntcgtcggcagcgtc

SEQ ID NO: 21 - DNA, 20 bp, synthetic construct
  source 1..20: mol_type = other DNA
acaagaggaagagagagacc

SEQ ID NO: 22 - DNA, 20 bp, synthetic construct
  source 1..20: mol_type = other DNA
cctcttcaaggggtctacat

SEQ ID NO: 23 - DNA, 22 bp, synthetic construct
  source 1..22: mol_type = other DNA
ggctttgaatgaaatacagatg

SEQ ID NO: 24 - DNA, 24 bp, synthetic construct
  source 1..24: mol_type = other DNA
agaagagtctatctttgaactagc

SEQ ID NO: 25 - DNA, 20 bp, synthetic construct
  source 1..20: mol_type = other DNA
gtaacctcactttcctgctc

SEQ ID NO: 26 - DNA, 20 bp, synthetic construct
  source 1..20: mol_type = other DNA
agcctgaaaattacccatcc